(19) United States
(12) Patent Application Publication
(10) Pub. No.: US 2021/0251698 A1
(43) Pub. Date: Aug. 19, 2021

(54) SYSTEM AND METHOD FOR MONITORING A DEVICE

(71) Applicant: Techssisted Surgical Pte Ltd, Singapore (SG)

(72) Inventors: Zenan Wang, Singapore (SG); Cong Fu, Singapore (SG); Zhen Lei, Singapore (SG); Chen Feng, Singapore (SG); Wei Tech Ang, Singapore (SG); Jiawei Mao, Singapore (SG); Xiaobin Gao, Singapore (SG)

(21) Appl. No.: 17/270,173

(22) PCT Filed: Nov. 12, 2019
(86) PCT No.: PCT/SG2019/050555

§ 371 Date: Feb. 22, 2021

(30) Foreign Application Priority Data
Sep. 12, 2018 (SG) 10201807900S

(51) Int. Cl. A61B 34/20; G02B 21/36

(52) U.S. Cl. CPC A61B 34/20; A61B 2034/2055; G02B 21/362

(57) ABSTRACT

The invention relates to a monitoring system for monitoring at least one device. The monitoring system comprises a magnification lens system to generate a magnified image of the at least one device, and at least three positional sensors in an arrangement around the magnification lens system to determine a position of the at least one device. The at least one device may be a surgical tool.

### TECHNICAL FIELD

Various embodiments relate to a monitoring system for monitoring a device and/or a further device. Various embodiments relate to a method of forming a monitoring system to monitor a device and/or a further device.

### BACKGROUND

Surgeries often involve making very small movement (or micromovement) of a device (e.g. surgical device) by hand. An example of a surgery may be the removal of blood clots from a retina in the treatment of retinal vein occlusion. This surgery involves an injection of anticoagulants (a kind of drug that stops blood from clotting) into tiny vessels of the retina. During such surgery, a surgeon has to be careful not to tear the vessels apart.

However, during surgery, a surgeon's accuracy and precision in manipulating a device may be limited by involuntary hand movements, which may even cause errors in surgery. Typical examples of involuntary hand movement are physiological oscillations, myoclonia and low-frequency drifts.

Physiological oscillations may cause the largest errors in surgery. Physiological oscillations may be defined as involuntary, approximately rhythmic, and roughly sinusoidal movement, having a peak-to-peak error that may exceed 100 μm. Physiological oscillations may be caused by mechanical factors as well as neuromuscular factors. Mechanical factors include vascular pulsation, room vibration and transmitted forces. Neuromuscular factors, on the other hand, are typically associated with the motor unit firing. Mechanical factors may be determined by limb stiffness or inertia and are susceptible to inertial loads as well as external spring. In contrast, neuromuscular factors are independent of limb stiffness or inertia and may have a frequency band of 8 to 12 Hertz (Hz).

During surgery, in particular microsurgery, the magnitudes of involuntary hand movement may be almost equal to the magnitudes of intentional hand movement, making it almost impossible to perform certain surgeries by hand alone. While physiological oscillations caused by mechanical factors may be attenuated by arm and wrist supports, neuromuscular factors may still give rise to physiological oscillations having a frequency of 8 to 12 Hz.

### SUMMARY

According to various embodiments, a monitoring system for monitoring at least one device may be provided. The monitoring system may include a magnification lens system configured to generate a magnified image of the at least one device. The monitoring system may further include at least three positional sensors in an arrangement around the magnification lens system. According to various embodiments, the positional sensors are configured to determine a position of the at least one device.

According to various embodiments, a surgical system may be provided. The surgical system may include a monitoring system and at least one device.

According to various embodiments, a method of forming a monitoring system to monitor at least one device may be provided. The method of forming the monitoring system may include providing a magnification lens system to generate a magnified image of the at least one device. The method of forming the monitoring system may further include providing at least three positional sensors in an arrangement around the magnification lens system to determine a position of the at least one device.

According to various embodiments, a method of forming a surgical system may be provided. The method of forming the surgical system may include providing a monitoring system and providing at least one device.

### BRIEF DESCRIPTION OF DRAWINGS

These and other features of the present inventive concept will become more apparent by describing in detail exemplary embodiments thereof with reference to the accompanying drawings of which:

FIG. 1 depicts a monitoring system for monitoring a device and/or a further device according to various embodiments;

FIG. 2A depicts a monitoring system for monitoring a device and/or a further device according to various embodiments;

FIG. 2B depicts a surgical system according to various embodiments;

FIG. 3A shows a perspective view of a surgical system according to various embodiments;

FIG. 3B shows a front view of the surgical system shown in FIG. 3A according to various embodiments;

FIG. 3C shows a top view of the surgical system shown in FIG. 3A according to various embodiments;

FIG. 3D shows a magnified view of the surgical system shown in FIG. 3A according to various embodiments;

FIG. 3E shows a side view of the surgical system shown in FIG. 3A according to various embodiments;

FIG. 3F shows a perspective view of the surgical system shown in FIG. 3A but with the controller arranged in a different orientation according to various embodiments;

FIG. 3G shows another side view of the surgical system shown in FIG. 3A according to various embodiments;

FIG. 3H shows a magnified see-through view of a circular support of the monitoring system shown in FIG. 3A according to various embodiments;

FIG. 4A shows a perspective view of a surgical system according to various embodiments;

FIG. 4B shows a side view of the surgical system shown in FIG. 4A according to various embodiments;

FIG. 4C shows a perspective view of the surgical system shown in FIG. 4A but with the stand arranged in a different orientation according to various embodiments;

FIG. 4D shows another side view of the surgical system shown in FIG. 4A according to various embodiments;

FIG. 4E shows a see-through view of a circular support of the monitoring system shown in FIG. 4A according to various embodiments;

FIG. 5A shows a perspective view of a portion of a monitoring system including the circular support, the plurality of sensor supports, the magnification lens system and a plurality of recesses according to various embodiments;

FIG. 5B shows a perspective view of a portion of a monitoring system including the circular support, the plurality of sensor supports, the magnification lens system and a plurality of carriages according to various embodiments;

FIG. 6 shows a perspective view of a positional sensor according to various embodiments;

FIG. 7A illustrates that when a device is in a first position and/or first orientation, each of at least two positional sensors may detect (or receive) a light emitted from each of the at least three light sources of the device according to various embodiments;

FIG. 7B illustrates that when a device is in a second position and/or orientation, only one positional sensor may detect (or receive) a light emitted from all three light sources of the device according to various embodiments;

FIG. 7C shows at least three positional sensors of a monitoring system according to various embodiments;

FIG. 7C illustrates that when a device is in a first position and/or first orientation, each of the at least three positional sensors may detect (or receive) a light emitted from each of the at least three light sources of the device according to various embodiments;

FIG. 7D illustrates that when a device is in a second position and/or orientation, where the body of the device may occlude (or block) a light of at least one light source from being detected by at least one positional sensor, at least two other positional sensors may detect (or receive) a light emitted from all three light sources of the device and thereafter determine a three-dimensional position and/or a three-dimensional orientation of the device according to various embodiments;

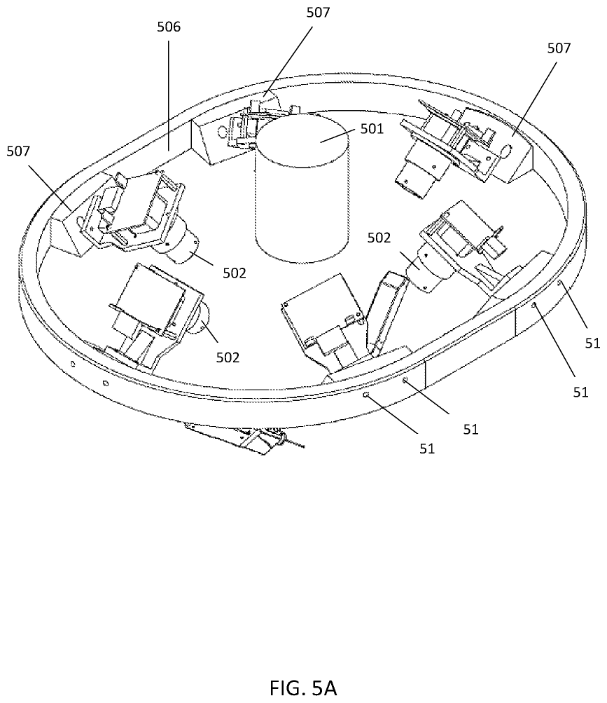

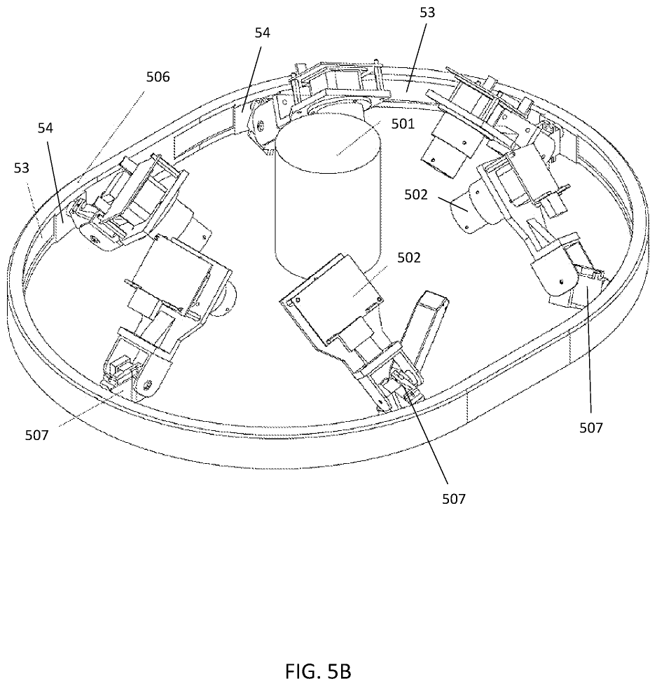

FIG. 5A shows a device and a further device, according to various embodiments;

FIG. 8B shows a time flow of action and inaction of a plurality of LEDs for calibration;

FIG. 9A shows an exploded view of a device according to various embodiments;

FIG. 9B shows an assembled view of a device shown in FIG. 9A according to various embodiments;

FIG. 9C shows an exterior view of a device shown in FIG. 9A according to various embodiments;

FIG. 9D shows a see-through view of a device according to various embodiments;

FIG. 9E illustrates the principle of the degrees of freedom along the x-axis and the y-axis of an end effector of a device according to various embodiments;

FIG. 9F illustrates the principle of the degrees of freedom along the x-axis and the y-axis of an end effector of a device, based on a x-y-axis frame, according to various embodiments;

FIG. 9G illustrates the principle of the degrees of freedom along the x-axis and the y-axis of an end effector of a device, based on the x-axis pin and the y-axis pin, according to various embodiments;

FIG. 9H illustrates the principle of the degrees of freedom along the z-axis of an end effector of a device according to various embodiments;

FIG. 9I shows a schematic side view of a device according to various embodiments;

FIG. 10A shows a perspective view of a motorized needle holder according to various embodiments;

FIG. 10B shows a side view of a motorized needle holder according to various embodiments;

FIG. 10C shows a view of a motorized needle holder of FIG. 10A according to various embodiments in which the clamping assembly part is separated from the motor assembly part;

FIG. 10D shows the clamping assembly part of a motorized needle holder according to various embodiments;

FIG. 10E shows the motor assembly part of a motorized needle holder according to various embodiments;

FIG. 10F shows a plurality of first forcep slices of a motorized needle holder according to various embodiments;

FIG. 10G shows a plurality of second forcep slices of a motorized needle holder according to various embodiments;

FIG. 11 shows a view of a disassembled motorized microsurgery scissors according to various embodiments;

FIG. 12A shows an exploded view of an injector (without a motor) according to various embodiments;

FIG. 12B shows a side view of an injector (without a motor) according to various embodiments;

FIG. 12C shows a cross-sectional side view of an injector (without a motor) according to various embodiments;

FIG. 12D shows a perspective view of a motorized injector according to various embodiments;

FIG. 12E shows a side view of a motorized injector according to various embodiments;

FIG. 12F shows a cross-sectional side view of a motorized injector according to various embodiments;

FIG. 12G shows a cross-sectional side view of a motorized injector according to various embodiments;

FIG. 12H shows an exploded view of a motorized injector according to various embodiments;

FIG. 12I shows a close-up view of a screw shaft of a motorized injector according to various embodiments;

FIG. 12J shows a close-up view of a piston-nut of a motorized injector according to various embodiments;

FIG. 13 is a schematic showing a method of forming a monitoring system according to various embodiments; and

FIG. 14 is a schematic showing a method of forming a surgical system according to various embodiments.

### DETAILED DESCRIPTION OF EXEMPLARY EMBODIMENTS

Embodiments described below in context of the apparatus are analogously valid for the respective methods, and vice versa. Furthermore, it will be understood that the embodiments described below may be combined, for example, a part of one embodiment may be combined with a part of another embodiment.

Various embodiments are provided for devices and/or systems, and various embodiments are provided for methods. It will be understood that basic features of the devices and/or systems also hold for the methods, vice versa. Therefore, for sake of brevity, duplicate description of such features may be omitted.

It will be understood that any feature described herein for a specific device may also hold for any device described herein. Accordingly, it will be understood that any feature described herein for a device may also hold for a further device described herein. It will be understood that any property described herein for a specific method may also hold for any method described herein.

It should be understood that the terms “on”, “over”, “top”, “bottom”, “down”, “side”, “back”, “left”, “right”, “front”, “lateral”, “side”, “up”, “down” etc., when used in the following description are used for convenience and to aid understanding of relative positions or directions, and not intended to limit the orientation of any device, structure or system, or any part of any device, structure or system. In addition, the singular terms “a”, “an”, and “the” include plural references unless the context clearly indicates otherwise. Similarly, the word “or” is intended to include “and” unless the context clearly indicates otherwise.

In the context of various embodiments, the articles “a”, “an” and “the” as used with regard to a feature or element include a reference to one or more of the features or elements.

In the context of various embodiments, the term “about” or “approximately” as applied to a numeric value encompasses the exact value and a reasonable variance.

As used herein, the term “and/or” includes any and all combinations of one or more of the associated listed items.

Unless specified otherwise, the terms “comprising”, “comprise”, “include” and “including”, and grammatical variants thereof, are intended to represent “open” or “inclusive” language such that they include recited elements but also permit inclusion of additional, unrecited elements.

Accordingly, example embodiments seek to provide a system that addresses at least some of the issues identified above.

Surgical robots have been designed to meet the demands in microsurgery and solve issues related to involuntary hand movement. One type of surgical robot is a ‘micromanipulator’.

There are generally three classes of micromanipulators: (i) Master & Slave, (ii) Co-operative and (iii) Handheld.

The Master & Slave class of micromanipulators includes micromanipulators that use tele-robotic technology. With tele-robotic technology, a user (e.g. surgeon) may use an input controller (i.e. Master) to control movement of a surgical instrument (i.e. Slave) that is located a distance away from both the user and the input controller. There are no mechanical linkages between the surgical instrument and the input controller. As such, micromanipulators of the Master & Slave class may be used in large spaces, and the Master and Slave components may be employed in various layouts. Micromanipulators of the Master & Slave class suppress involuntary hand movement by modifying and filtering signals sent from the input controller to the surgical instrument. However, micromanipulators of the Master & Slave class may have a high latency, may have a lack (or absence) of a tactile feedback (or physical force feedback) to the user and may come with a high price tag. Micromanipulators of the Master & Slave class are also often very expensive. Further, some micromanipulators of the Master & Slave class, for example, da Vinci robot, is designed for general surgeries and not for microsurgeries.

The Co-operative class of micromanipulators combines the respective inputs of a user and a robot. With the Co-operative class of micromanipulators, a user manipulates a device and, at the same time, a robot with a filter (e.g. mechanical low-pass filter) cancels involuntary hand movement of the user by way of the filter. The robot provides added stiffness to the device, rendering the device less susceptible to involuntary hand movement. However, the stiffness provided by the robot may result in sluggish movement of the device, and the robot usually does not provide a tactile feedback to the user. An example of a micromanipulator in the Co-operative class is the Steady-Hand Eye-Robot from Johns Hopkins University.

The Handheld class of micromanipulators may include a handheld device, having a control module embedded within the device. External Position Sensitive Detectors (PSDs) may be used to detect a position of the device through a vision tracking system within a workspace. However, in order to ensure the signals emitted from the handheld device are detected, the line of sight of each PSD must not be occluded. Accordingly, the number of possible poses (e.g. working poses) and workspace of a handheld device (e.g. a micromanipulator) may be limited. An example of a micromanipulator in the Handheld class is Micron, developed by Riveiere et al. from Carnegie Mellon University. Micron utilizes a normal binocular vision system with only two PSDs. The two PSDs are mounted beside a table and are fixed in position (or immovable, unadjustable), such that the optical axes of the two PSDs are orthogonal with each other. Accordingly, a workspace (e.g. location for placing a device for monitoring of the device) of Micron is fixed. Also, Micron may only detect a limited number of poses of a device, since for certain poses of the device or certain poses of a user's hand (holding the device), the line of sight of at least one of the two PSDs (to the device) may be occluded. Further, Micron does not include a microscope, which may be necessary during certain surgeries. Accordingly, if a microscope is utilized together with Micron, the Micron system is separate from the microscope, and a user may not move the immovable micron system or change the line of sight of the two PSDs, when the user moves the microscope.

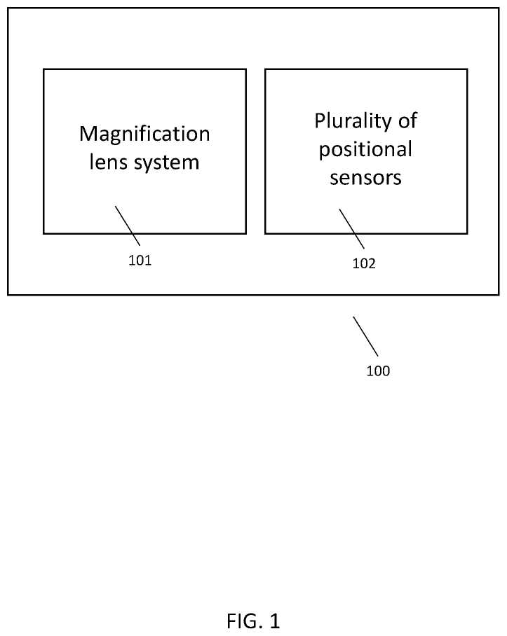

FIG. 1 depicts a monitoring system 100 for monitoring a device and/or a further device according to various embodiments. The monitoring system 100 may include a magnification lens system 101 configured to generate a magnified image of the device (e.g. magnified image of a tooltip of the device) that is positioned within a field of view of the magnification lens system 101. For example, when the device is positioned in front of a lens of the magnification lens system 101, the magnification lens system 101, when in operation, may capture an image of the device and thereafter generate a magnified image of the device.

According to various embodiments, the magnification lens system 101 may be a microscope, for example, an optical microscope or a digital microscope. According to various embodiments, the magnification lens system 101 may be a lens (e.g. magnifying lens).

The monitoring system 100 may further include a plurality of positional sensors 102 which may be positioned in an arrangement around or surrounding the magnification lens system 101. According to various embodiments, the arrangement may be a circular-shaped arrangement (or circular arrangement). It may be envisioned that according to various embodiments, the circular-shaped arrangement may be a circle-shaped arrangement (circle arrangement), an oval-shaped arrangement (or oval arrangement), an ellipse-shaped arrangement (or ellipse arrangement) or any other suitably-shaped arrangement around or surrounding the magnification lens system 101.

According to various embodiments, the plurality of positional sensors 102 may include any suitable number of positional sensors, for example, at least three positional sensors (e.g. three or more than three positional sensors). According to various embodiments, the plurality of positional sensors 102 may be a plurality of position sensor detectors (PSDs), for example, at least three PSDs (e.g. three or more than three PSDs). According to various embodiments, the plurality of positional sensors 102 may be a plurality of infrared sensors, a plurality of optical sensors, a plurality of any suitable optical sensors or a combination of different types of suitable optical sensors.

The plurality of positional sensors 102 may be configured to determine a position and/or an orientation of a device (and/or a further device). For example, when the device is positioned in front of a lens of one positional sensor of the plurality of positional sensors 102, the positional sensor, when in operation, may detect (e.g. capture or receive) a light (e.g. wanted light signal(s) or filtered light signal(s)) emitted from a light source of the device and thereafter determine a position of the light source and, in turn, a position of the device (e.g. a position of a portion of the device where the light source is disposed or located) based on the detected (or received) light. According to various embodiments, the device may include a plurality of light sources, for example, embedded on a surface of the device at a predetermined portion (e.g. handle) of the device. According to various embodiments, the device may include at least three light sources which may be, for example, equilaterally spaced apart from one another around a tubular surface of a handle of the device or positioned at any portion on a surface of the device. Accordingly, according to various embodiments, the plurality of positional sensors 102 of the monitoring system 100 may be configured to detect a light (e.g. wanted light signal(s) or filtered light signal(s)) emitted from each of the at least three light sources of the device and thereafter determine a three-dimensional position and/or a three-dimensional orientation of the device (e.g. of a portion of the device where the at least three light sources are disposed or located on the device). For example, when at least two positional sensors of the plurality of positional sensors 102 (e.g. at least three positional sensors) detect (or receive) a light emitted from each of the at least three light sources of the device, the at least two positional sensors may determine a three-dimensional position and/or a three-dimensional orientation of the device. In this disclosure, “three-dimensional position” may refer to a three-dimensional position of a subject (e.g. object or device) in space and “three-dimensional orientation” may refer to a three-dimensional orientation of a subject in space.

Accordingly, according to various embodiments, the monitoring system may determine a position (e.g. three-dimensional position) and/or an orientation (e.g. three-dimensional orientation) of the device (e.g. geometric feature of the device, such as edge, corner, shape etc. of the device) based on detection, by at least two of at least three positional sensors (e.g. PSDs), of light emitted from a plurality of light sources (e.g. at least three light sources) of the device.

According to various embodiments, a light from the light source or from the plurality of light sources (e.g. at least three light sources) of the device may be a visible light, an infrared light etc., or any other suitable light. According to various embodiments, the device may be a handheld device.

It may also be envisioned that in various embodiments, at least one reflective surface (e.g. retro-reflective material/market, mirror etc.) may replace the light source or the plurality of light sources of the device. For example, the at least one reflective surface may be at least three reflective surfaces which may be, for example, equilaterally spaced apart from one another around a tubular surface of a handle of the device or positioned at any portion on a surface of the device. Accordingly, the at least one reflective surface may be configured to reflect a light from a light source (e.g. light source external to the device or ambient light).

Accordingly, according to various embodiments, the monitoring system may determine a position (e.g. three-dimensional position) and/or an orientation (e.g. three-dimensional orientation) of the device (e.g. geometric feature of the device, such as edge, corner, shape etc. of the device) based on detection, by at least two of at least three positional sensors (e.g. PSDs), of light reflected from a plurality of reflective surfaces (e.g. at least three reflective surfaces) of the device.

It may also be envisioned that in various embodiments, at least one reflective surface (e.g. retro-reflective material) may be additionally coated to a surface of the device having the light source or the plurality of light sources. Accordingly, the at least one reflective surface that is coated on the device may be configured to reflect a light, and the light source or the plurality of light sources may be configured to emit a light.

Accordingly, according to various embodiments, the monitoring system may determine a position (e.g. three-dimensional position) and/or an orientation (e.g. three-dimensional orientation) of the device (e.g. geometric feature of the device, such as edge, corner, shape etc. of the device) based on detection, by at least two of at least three positional sensors (e.g. PSDs), of light reflected from the at least one reflective surface (e.g. retro-reflective material/market, mirror etc.) and/or of light emitted from the light source or the plurality of light sources (e.g. at least three light sources) of the device.

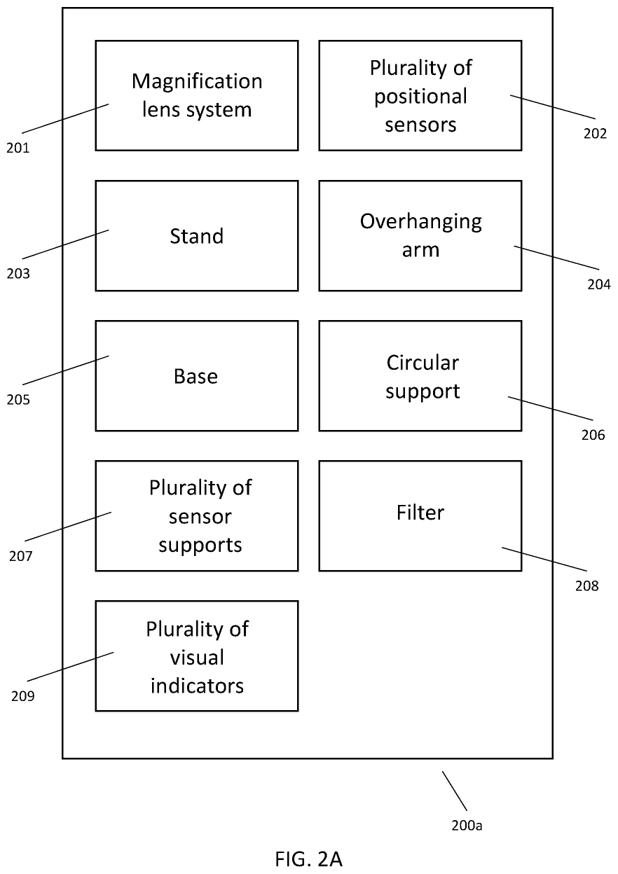

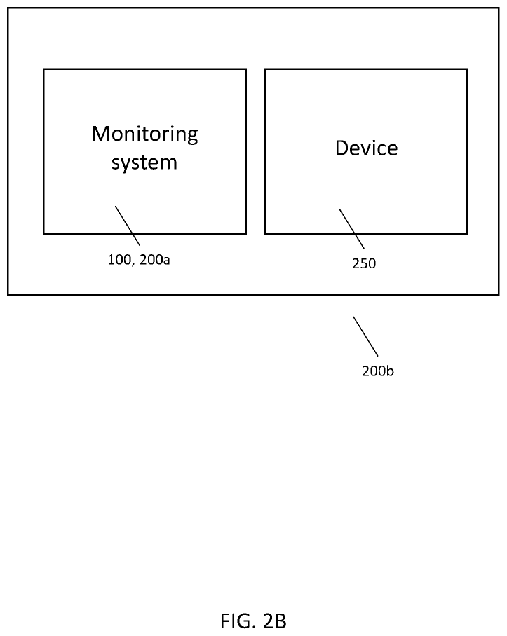

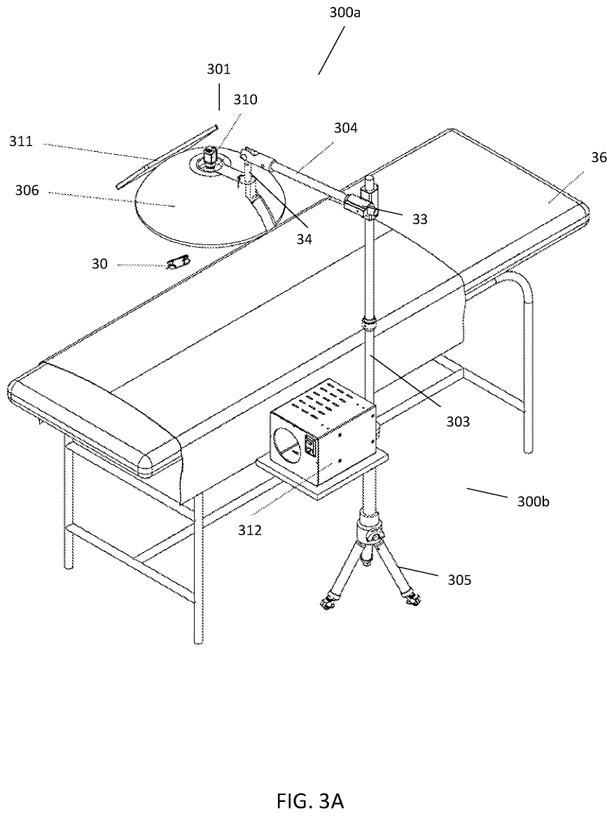

FIG. 2A depicts a monitoring system 200a for monitoring a device and/or a further device according to various embodiments.

The monitoring system 200a may, similar to FIG. 1, include a magnification lens system 201 configured to generate a magnified image of the device (e.g. magnified image of a tooltip of the device) that is positioned within a field of view of the magnification lens system 201. For example, when the device is positioned in front of a lens of the magnification lens system 201, the magnification lens system 201, when in operation, may capture an image of the device and thereafter generate a magnified image of the device. According to various embodiments, the magnification lens system 201 may be a microscope, for example, an optical microscope or a digital microscope. According to various embodiments, the magnification lens system 201 may be a lens (e.g. magnifying lens).

The monitoring system 200a may, similar to FIG. 1, further include a plurality of positional sensors 202 which may be positioned in an arrangement around or surrounding the magnification lens system 201. According to various embodiments, the arrangement may be a circular-shaped arrangement (or circular arrangement). It may be envisioned that according to various embodiments, the circular-shaped arrangement may be a circle-shaped arrangement (circle arrangement), an oval-shaped arrangement (or oval arrangement), an ellipse-shaped arrangement (or ellipse arrangement) or any other suitably-shaped arrangement around or surrounding the magnification lens system 201. According to various embodiments, the plurality of positional sensors 202 positioned in an arrangement around or surrounding the magnification lens systems 201 may refer to the plurality of positional sensors 202 positioned around or surrounding an area along an optical axis of the magnification lens system 201.

According to various embodiments, the plurality of positional sensors 202 may include any suitable number of positional sensors, for example, at least three positional sensors (e.g. three or more than three positional sensors). Having three or more positional sensors may address sightline occlusion. Further, according to various embodiments, the plurality of positional sensors 202 may be a plurality of PSDs, for example, at least three PSDs (e.g. three or more than three PSDs). According to various embodiments, the plurality of positional sensors 202 may be a plurality of infrared sensors, a plurality of optical sensors, a plurality of any suitable optical sensors, or a combination of different types of suitable optical sensors.

The plurality of positional sensors 202 may be configured to determine a position and/or an orientation of a device. For example, when the device is positioned in front of a lens of one positional sensor of the plurality of positional sensors 202, the positional sensor, when in operation, may detect (e.g. capture or receive) a light (e.g. wanted light signal(s) or filtered light signal(s)) emitted from a light source of the device and thereafter determine a position of the light source and, in turn, a position of the device (e.g. a position of a portion of the device where the light source is disposed) based on the detected (or received) light. According to various embodiments, the device may include a plurality of light sources, for example, on a surface of the device at a predetermined portion (e.g. handle) of the device. According to various embodiments, the device may include at least three light sources, for example, equilaterally spaced apart on the surface of the device. Accordingly, according to various embodiments, the plurality of positional sensors 202 of the monitoring system 200a may be configured to detect a light (e.g. wanted light signal(s) or filtered light signal(s)) emitted from each of the at least three light sources of the device and thereafter determine a three-dimensional position and/or a three-dimensional orientation of the device (e.g. of a portion of the device where the at least three light sources are disposed on the device). For example, when at least two positional sensors of the plurality of positional sensors 202 (e.g. at least three positional sensors) detect (or receive) a light emitted from each of at least three light sources of the device, the at least two positional sensors may determine a three-dimensional position and/or a three-dimensional orientation of the device.

Accordingly, according to various embodiments, the monitoring system may determine a position (e.g. three-dimensional position) and/or an orientation (e.g. three-dimensional orientation) of the device (e.g. geometric feature of the device, such as edge, corner, shape etc. of the device) based on detection, by at least two of at least three positional sensors (e.g. PSDs), of light emitted from a plurality of light sources (e.g. at least three light sources) of the device.

According to various embodiments, the light from a light source or from a plurality of light sources (e.g. at least three light sources) of the device may be a visible light, an infrared light etc., or any other suitable light.

It may also be envisioned that in various embodiments, at least one reflective surface (e.g. retro-reflective material/market, mirror etc.) may replace the light source or the plurality of light sources of the device. For example, the at least one reflective surface may be at least three reflective surfaces which may be, for example, equilaterally spaced apart from one another around a tubular surface of a handle of the device or positioned at any portion on a surface of the device. Accordingly, the at least one reflective surface may be configured to reflect a light from a light source (e.g. light source external to the device or ambient light).

Accordingly, according to various embodiments, the monitoring system may determine a position (e.g. three-dimensional position) and/or an orientation (e.g. three-dimensional orientation) of the device (e.g. geometric feature of the device, such as edge, corner, shape etc. of the device) based on detection, by at least two of at least three positional sensors (e.g. PSDs), of light reflected from a plurality of reflective surfaces (e.g. at least three reflective surfaces) of the device.

It may also be envisioned that in various embodiments, at least one reflective surface (e.g. retro-reflective material) may be additionally coated to a surface of the device having the light source or the plurality of light sources. Accordingly, the at least one reflective surface that is coated on the device may be configured to reflect a light, and the light source or the plurality of light sources may be configured to emit a light.

Accordingly, according to various embodiments, the monitoring system may determine a position (e.g. three-dimensional position) and/or an orientation (e.g. three-dimensional orientation) of the device (e.g. geometric feature of the device, such as edge, corner, shape etc. of the device) based on detection, by at least two of at least three positional sensors (e.g. PSDs), of light reflected from the at least one reflective surface (e.g. retro-reflective material/market, mirror etc.) and/or of light emitted from the light source or the plurality of light sources (e.g. at least three light sources) of the device.

According to various embodiments, the monitoring system 200a may further include a stand 203. The stand 203 may include a first end portion and a second end portion.

According to various embodiments, the monitoring system 200a may further include an overhanging arm 204 extending from the first end portion of the stand 203.

According to various embodiments, the overhanging arm 204 may be detachably coupled to the first end portion of the stand 203 by any suitable means.

Alternatively, the overhanging arm 204 and the stand 203 may be a single integral structure.

According to various embodiments, the magnification lens system 201 and the plurality of positional sensors 202 (e.g. at least three positional sensors or at least three PSDs) may be attached to the overhanging arm 204. According to various embodiments, the magnification lens system 201 and the plurality of positional sensors 202 may move in tandem. For example, the plurality of positional sensors 202 may move together with the magnification lens system 201 (e.g. microscope) whenever the magnification lens system 201 is moved by a user.

Alternatively, according to various embodiments, the magnification lens system 201 and the plurality of positional sensors 202 may move independently of one another. For example, according to various embodiments, the magnification lens system 201 may be attached to an operating table and the plurality of positional sensors 202 may be attached to the overhanging arm 204 such that magnification lens system 201 and the plurality of positional sensors 202 may move independently of one another.

According to various embodiments, the monitoring system 200a may further include a base 205 attached to the second end portion of the stand 203. The base 205 may be detachably coupled to the second end portion of the stand 203 by any suitable means.

Alternatively, the base 205 and the stand 203 may be a single integral structure.

According to various embodiments, the base 205 may be configured to support and hold the stand 203, including the overhanging arm 204 extending from the first end portion of the stand 203, in an upright position. In other words, the base 205 may be configured to prevent the stand 203, including the overhanging arm 204 extending from the first end portion of the stand 203, from toppling, when the stand 203 is in the upright position.

According to various embodiments, the monitoring system 200a may further include a circular support 206 connected to the overhanging arm 204. The circular support 206 may be configured to hold the plurality of positional sensors 202 (e.g. at least three positional sensors or at least three PSDs) in an arrangement around or surrounding the magnification lens system 201. According to various embodiments, the magnification lens system 201 may be coupled to the plurality of positional sensors 202 via the circular support 206 or by any suitable means.

It may also be envisioned that according to various embodiments, at least one of the magnification lens system 201, the plurality of positional sensors 202 (e.g. at least three positional sensors or at least three PSDs) and the circular support 206 may be attached to any suitable surface or support (e.g. overhanging surface/support or ceiling, wall etc.) such that the monitoring system 200a may be operable without the need for at least the stand 203 and/or the overhanging arm 204. For example, the magnification lens system 201, the plurality of positional sensors 202 and the circular support 206 may be coupled to one another and the circular support 206 may, in turn, be attached to and/or hang from a suitable surface or support (e.g. overhanging surface/support or ceiling, wall etc.).

As an example, the arrangement of the plurality of positional sensors 202 around or surrounding the magnification lens system 201 may be a circular-shaped arrangement (or circular arrangement). It may be envisioned that according to various embodiments, the circular-shaped arrangement may be a circle-shaped arrangement (circle arrangement), an oval-shaped arrangement (or oval arrangement), an ellipse-shaped arrangement (or ellipse arrangement) or any other suitably-shaped arrangement around or surrounding the magnification lens system 201. Accordingly, the circular support 206 may have a shape including a circle, an oval, an ellipse or any other suitable shape.

According to various embodiments, the circular support 206 may be an annular structure. The annular structure may be a wall. The wall may have an inner surface and an outer surface opposite the inner surface. The inner surface of the wall may define a through-hole, which may be or may include the center of the circular support 206 (i.e. annular structure). For example, the circular support 206 may be a rail, for example, an annular rail having an inner wall and an outer wall, and the inner wall may define a through-hole, which may be or may include the center of the annular rail. The magnification lens system 201 may be coupled to the overhanging arm 204 and may be positioned along a longitudinal axis of the through-hole that defines the center of the circular support 206 (i.e. annular rail), for example, as shown in FIGS. 5A-5B.

Alternatively, the circular support 206 may be a closed structure (i.e. without any through-hole), and the magnification lens system 201 may be coupled to any suitable portion of the circular support 206.

According to various embodiments, the circular support 206 may include a plurality of components which form the circular support 206.

According to various embodiments, each component of the plurality of components may include a symmetric shape.

According to various embodiments, each component may have a similar or identical shape to another (e.g. neighboring) component of the plurality of components.

According to various embodiments, all the components of the plurality of components may have an identical shape.

According to various embodiments, the plurality of components of the circular support 206 may be coupled (or joined) to one another by any suitable means to form the circular support 206. Accordingly, when the circular support 206 is an annular rail, the circular support 206 may include a plurality of rail components (e.g. two semi-circular rail components) which may be coupled together to form the circular support 206.

According to various embodiments, the monitoring system 200a may further include a plurality of sensor supports 207 attached to the circular support 206, the plurality of sensor supports configured to hold the plurality of positional sensors 202. The plurality of position sensors 202 may be held by the circular support 206 via the plurality of sensor supports 207. Accordingly, the plurality of sensor supports 207 may be attached to the circular support 206 in an arrangement around or surrounding the magnification lens system 201, to enable the plurality of positional sensors 202 to be in the arrangement around or surrounding the magnification lens system 201.

Each sensor support of the plurality of sensor supports 207 may be attached to a respective predetermined portion of the circular support 206 via a coupling (e.g. a first coupling) between each sensor support and the respective predetermined portion of the circular support 206.

According to various embodiments, the circular support 206 may include a plurality of predetermined portions for receiving the plurality of sensor supports 207, and each sensor support of the plurality of sensor supports 207 may be interchangeably attached to a respective predetermined portion of the circular support 206 for receiving the plurality of sensor supports 207 (and corresponding plurality of positional sensors 202).

According to various embodiments, the coupling (i.e. first coupling) between each sensor support and the circular support 206 may include a mechanical fastener. For example, the mechanical fastener may include a nail, a screw (e.g. a threaded screw), a bolt (e.g. bolt and nut), a stud, a rivet, or other suitable types of mechanical fastener. Accordingly, a sensor support of the plurality of sensor supports 207 may be attached to a predetermined portion of the circular support 206 (e.g. to a portion of an inner surface of a wall of an annular rail or to any portion of any suitable type of circular support 206), via the coupling, such that the sensor support is immovable relative to the predetermined portion of the circular support 206.

For example, each predetermined portion of the circular support 206 for receiving the plurality of sensor supports 207 may include at least one recess (e.g. mechanical fastener recess, a threaded through-hole or any suitable recess for receiving a mechanical fastener) for receiving a first portion of a mechanical fastener therethrough. Further, each sensor support may include at least one recess (e.g. from a surface of the sensor support configured to abut the circular support 206) for receiving a second portion of the mechanical fastener. When the at least one recess (or a recess of the at least one recess) of the circular support 206 is aligned with the at least one recess (or a recess of the at least one recess) of the sensor support (i.e. the sensor support in question), a mechanical fastener may be inserted through the aligned recesses to immovably attach the sensor support to the circular support 206.

According to various embodiments, each sensor support of the plurality of sensor supports 207 may be magnetically coupled or attached to the circular support 206 (e.g. a dome or annular rail) by any suitable means, such that each sensor support of the plurality of sensor supports 207 may be positioned on (e.g. manually) and attached to any suitable surface of the circular support 207 via magnetic coupling.

According to various embodiments, each sensor support of the plurality of sensor supports 207 may be slidably coupled or attached to the circular support 206 by any suitable means.

For example, the circular support 206 may be an annular rail and each sensor support of the plurality of sensor supports 207 may be attached to a carriage that is, in turn, slidably coupled or attached to the annular rail such that the carriage (and the corresponding sensor support attached thereto) may be movable (e.g. slidable) along the annular rail. Accordingly, the carriage may be movable (e.g. manually or by motorized means) along the annular rail, and once a desired (or predetermined) position of the carriage is determined, the carriage may be immovably held in the determined (e.g. desired) position by any suitable means (e.g. using a stopper, a mechanical fastener or coupler).

According to various embodiments, the plurality of sensor supports 207 may be configured to move (e.g. slide) along the annular rail (i.e. circular support 206) by motorized means.

According to various embodiments, the motorized means may be configured to move the plurality of sensor supports 207 in tandem (e.g. simultaneously), relative to the annular rail. For example, the annular rail may include a belt drive motor and a conveyor belt. One side (e.g. surface) of the conveyor belt may be coupled to the belt drive motor. On another side of the conveyor belt, a plurality of sensor supports 207 may be attached to respective portions of said side of the conveyor belt. Accordingly, driving the belt drive motor may cause the conveyor belt, and the plurality of sensor supports 207 attached thereto, to move in tandem.

Alternatively, the motorized means may be configured to move each sensor support of the plurality of sensor supports 207 relative to the annular rail individually. In other words, the motorized means may be configured to move each sensor support of the plurality of sensor supports 207 independently of one another. For example, a plurality of carriages may be slidably coupled or attached to the annular rail (i.e. circular support 206). Each sensor support of the plurality of sensor supports 207 may be attached to a respective carriage of a plurality of carriages. Further, each carriage may include a motor configured to move the carriage (and the corresponding sensor support attached thereto), individually (or independently), relative to the annular rail.

According to various embodiments, the circular support 206 may include a scale (e.g. grating scale). For example, the circular support 206 may be an annular rail with a scale on a surface of the annular rail (i.e. circular support 206), for example, on an inner surface of a wall and/or an outer surface of the wall of the annular rail.

According to various embodiments, the scale of the circular support 206 may be configured to detect (or determine) a position of each sensor support (and corresponding positional sensor) on the annular rail (i.e. circular support 206) relative to the circular support 206 and may further be configured to provide information of the position of each sensor support of the plurality of sensor supports 207 relative to the circular support 206. It may also be envisioned that in various embodiments, any suitable encoder may be used to detect and determine a position of each sensor support of a plurality of sensor supports 207 on the circular support 206.

According to various embodiments, each sensor support of the plurality of sensor supports 207 may be attached to one positional sensor of the plurality of positional sensors 202 to hold the positional sensor. In other words, each sensor support of the plurality of sensor supports 207 may be configured to hold one positional sensor of the plurality of positional sensors, for example, via a coupling (e.g. a second coupling) between the sensor support and the positional sensor. According to various embodiments, each positional sensor of the plurality of positional sensors 202 may be interchangeably attached to each sensor support of the plurality of sensor supports 207. In other words, according to various embodiments, the plurality of sensor supports 207 and the plurality of positional sensors 202 may be interchangeably attached to one another.

According to various embodiments, the coupling (i.e. second coupling) between the sensor support and the positional sensor may include a mechanical fastener. For example, the mechanical fastener may include a nail, a screw (e.g. a threaded screw), a bolt (e.g. bolt and nut), a stud, a rivet, or other suitable types of mechanical fastener. According to various embodiments, the coupling between each sensor support and each positional sensor of the plurality of positional sensors 202 may be manipulated to allow a position (e.g. front, back, up, down, left or right position) and/or an orientation (e.g. an elevation angle, an azimuth angle and/or a torsion angle) of each positional sensor to be adjusted relative to a respective sensor support. Accordingly, a position and/or an orientation of a positional sensor may be adjusted (e.g. manually), and once a desired orientation and/or position of the positional sensor is determined, the positional sensor may be attached to the respective sensor support and held firmly (or rigidly) in place via the coupling. For example, the mechanical fastener may be loosened (e.g. to loosely couple a positional sensor to a respective sensor support) to allow a position and/or an orientation of the positional sensor to be adjusted. Further, the mechanical fastener may be tightened to firmly secure the positional sensor to the respective sensor support (such that the positional sensor is immovable relative to the sensor support).

According to various embodiments, the coupling (i.e. second coupling) between the sensor support and the positional sensor may include a flexible structure including a shape-retaining material. For example, the coupling may be a wire (e.g. metal wire, alloy wire, nitinol wire etc.). Accordingly, when the coupling includes a flexible structure including a shape-retaining material, a position and/or an orientation (e.g. an elevation angle, an azimuth angle and/or a torsion angle) of the positional sensor relative to a respective sensor support may be adjusted (e.g. by manual manipulation) and held in place (e.g. when no adjustment or manipulation is made) by way of, at least, manipulation of the flexible structure including the shape-retaining material.

According to various embodiments, the coupling between each sensor support and each respective positional sensor may include at least one motor configured to move and adjust a position (e.g. front, back, up, down, left or right position) and/or an orientation (e.g. an elevation angle, an azimuth angle and/or a torsion angle) of the positional sensor relative to the respective sensor support.

According to various embodiments, a position and/or an orientation (e.g. angular rotation) of each positional sensor relative to each respective sensor support may be detected and determined by an encoder.

According to various embodiments, the monitoring system 200a may further include a filter 208. According to various embodiments, the filter 208 may be incorporated within each of the plurality of positional sensors 202. The filter 208 may be configured to filter out a predetermined spectrum of light (e.g. unwanted light signal(s)), for example, visible light, and allow another predetermined spectrum of light (e.g. wanted light signal(s) or filtered light signal(s)), for example, infrared light, to be detected (or received) by the plurality of positional sensors. In other words, the filter 208 may block a spectrum of light (e.g. unwanted light signal(s)) from being detected (or received) by the plurality of positional sensors 202 and allow another spectrum of light (e.g. wanted light signal(s) or filtered light signal(s)) to be detected by the plurality of positional sensors 202. Accordingly, according to various embodiments, the filter 208 may be configured to allow a specified (or predetermined e.g. by a user) wavelength signal (e.g. of light) or a specified range of wavelength signals (e.g. from active marker(s) such as light source(s) and/or passive marker(s) such as reflective surface(s)) to be recognized (e.g. detected or captured) by a corresponding positional sensor of the plurality of positional sensors 202, and the filter 208 may be configured to filter out or block a predetermined wavelength signal or a predetermined range of wavelength signals from being detected by the corresponding positional sensor of the plurality of positional sensors 202. In this specification, reference to “specified wavelength signal” or “specified range of wavelength signals” may be reference to wanted light signal(s) or filtered light signal(s) (e.g. as desired by a user) to be detected (e.g. captured or received) by positional sensor(s), according to various embodiments. Accordingly, according to various embodiments, each positional sensor of the plurality of positional sensors 202 may be configured to filter out a predetermined wavelength signal (e.g. of light) or a predetermined range of wavelength signals (e.g. of light).

According to various embodiments, the monitoring system 200a may further include a plurality of visual indicators 209, wherein at least one visual indicator of the plurality of visual indicators 209 may be attached to each positional sensor of the plurality of positional sensors 202 and may be further configured to provide a visual indication of a working space of the respective positional sensor to which the at least one visual indicator is attached to.

FIG. 2B depicts a surgical system 200b according to various embodiments.

The surgical system 200b may include a monitoring system 100, 200a according to various embodiments.

The surgical system 200b may further include a device 250, which may be the device mentioned with reference to FIGS. 1 and 2A, according to various embodiments.

According to various embodiments of the surgical system 200b, the device 250 may include a plurality of light sources (e.g. at least three light sources), for example, embedded on a surface of the device 250 at a predetermined portion (e.g. handle) of the device 250. According to various embodiments, the device 250 may include at least three light sources (e.g. four or more) which may be, for example, equilaterally spaced apart from one another around a tubular surface of a handle of the device 250 or positioned at any portion on a surface of the device 250.

According to various embodiments of the surgical system 200b, a position (e.g. three-dimensional position) and/or an orientation (e.g. three-dimensional position) of the device 250 (e.g. geometric feature of the device 250, such as edge, corner, shape etc. of the device 250) may be determined (e.g. by at least two of at least three positional sensors of the monitoring system) based on light emitted from the plurality of light sources of the device 250 (e.g. at least three light sources).

According to various embodiments of the surgical system 200b, the device 250 is a surgical tool, for example, a holder, a single-blade cutter (e.g. knife), a dual-blade cutter (e.g. scissors), a fluid injector (e.g. syringe) or any other suitable surgical tool.

According to various embodiments, the surgical system 200b may further include a further device.

According to various embodiments of the surgical system 200b, the further device may include a plurality of light sources (e.g. at least three light sources), for example, embedded on a surface of the further device at a predetermined portion (e.g. handle) of the further device. According to various embodiments, the further device may include at least three light sources which may be, for example, equilaterally spaced apart from one another around a tubular surface of a handle of the further device or positioned at any portion on a surface of the further device.

According to various embodiments of the surgical system 200b, a position (e.g. three-dimensional position) and/or an orientation (e.g. three-dimensional position) of the further device (e.g. geometric feature of the further device, such as edge, corner, shape etc. of the further device) may be determined (e.g. by at least two of at least three positional sensors of the monitoring system) based on light emitted from the plurality of light sources of the further device (e.g. at least three light sources).

According to various embodiments of the surgical system 200b, the further device is a surgical tool, for example, a holder, a single-blade cutter (e.g. knife), a dual-blade cutter (e.g. scissors), a fluid injector (e.g. syringe) or any other suitable surgical tool.

According to various embodiments, the monitoring system 100, 200a or the surgical system 200b may further include an image detector (or camera) configured to detect a magnified image generated by a magnification lens system of the monitoring system 100, 200a.

According to various embodiments, the monitoring system 200a or the surgical system 200b may further include a monitor coupled to the image detector, the monitor configured to display the magnified image generated by the magnification lens system.

According to various embodiments, the monitoring system 100, 200a or the surgical system 200b may further include a computer coupled to the plurality of positional sensors 102, 202 (e.g. at least three positional sensors).

According to various embodiments, the monitoring system 100, 200a or the surgical system 200b may further include a controller coupled to the computer, the controller further coupled to the device 250 and/or to the further device. The controller may be further coupled to the plurality of positional sensors 102, 202 and/or couplings which connect the plurality of positional sensors 102, 202 to a circular support.

According to various embodiments, the computer may be configured to receive data on a position (e.g. three-dimensional position) and/or an orientation (e.g. three-dimensional orientation) of the device 250 and/or the further device determined by at least two positional sensors of the plurality of positional sensors 102, 202 (e.g. at least three positional sensors). In other words, the plurality of positional sensors 102, 202 may be configured to transmit data on the position (e.g. three-dimensional position) and/or the orientation (e.g. three-dimensional orientation) of the device 250 and/or the further device to the computer. Data on the position and/or orientation of the device 250 may be referred to as a “first data”, and data on the position and/or orientation of the further device may be referred to as a “second data”.

According to various embodiments, the computer may be configured to transmit data (i.e. data received from the plurality of positional sensors) on the position (e.g. three-dimensional position) and/or the orientation (e.g. three-dimensional orientation) of the device 250 and/or data on the position and/or the orientation of the further device to the controller. In other words, the controller may be configured to receive data on the position (e.g. three-dimensional position) and/or the orientation (e.g. three-dimensional orientation) of the device 250 and/or the further device from the computer.

According to various embodiments, the controller may be configured to control the device based on data (or feedback data, i.e. data received from the computer and the plurality of positional sensors) on the position (e.g. three-dimensional position) and/or the orientation (e.g. three-dimensional orientation) of the device. According to various embodiments, the controller may be further configured to control the further device based on data on the position and/or the orientation of the further device.

Further, according to various embodiments, the controller may be configured to control (e.g. move) a position (e.g. front, back, up, down, left or right position) and/or an orientation (e.g. an elevation angle, an azimuth angle and/or a torsion angle) of at least one positional sensor (of a plurality of positional sensors) relative to a respective sensor support and/or a position of the at least one positional sensor relative to a circular support to which the at least one positional sensor (and respective at least one sensor support) is attached thereto (e.g. changing the position of the positional sensor, from a first position on the circular support to any other position along the circular support), based on data (or feedback data, i.e. received from the computer and the plurality of positional sensors) on the position (e.g. three-dimensional position) and/or the orientation (e.g. three-dimensional orientation) of the device.

According to various embodiments, the controller may be configured to control (e.g. move) a position (e.g. front, back, up, down, left or right position) and/or an orientation (e.g. an elevation angle, an azimuth angle and/or a torsion angle) of at least one positional sensor (of a plurality of positional sensors) relative to a respective sensor support and/or a position of the at least one positional sensor relative to a circular support to which the at least one positional sensor (and respective at least one sensor support) is attached thereto, based on data (or feedback data, i.e. received from the computer and the plurality of positional sensors) on the position (e.g. three-dimensional position) and/or the orientation (e.g. three-dimensional orientation) of the further device.

In other words, according to various embodiments of the monitoring system 200a or the surgical system 200b, the controller may be configured to control (e.g. move) the device and/or the further device and/or a positional sensor and/or the plurality of positional sensors, based on the data of the three-dimensional position and/or a three-dimensional orientation of the device and/or the further device.

For example, based on data of a three-dimensional position and/or a three-dimensional orientation of the device, the computer may determine and generate data on a magnitude (e.g. scalar quantity) and/or vector (including magnitude and direction) of involuntary hand movement from the hand that is manipulating or handling the device. The computer may transmit the generated data to a controller. The controller may then provide a counter force (of a magnitude and/or vector opposite to the involuntary hand movement) to the device, e.g. on a tooltip or an end effector of the device, to attenuate the involuntary hand movement from the hand to the device, based on the data generated and transmitted by the computer.

Similarly, based on the data of the three-dimensional position and/or the three-dimensional orientation of the further device, the computer may determine and generate data on a magnitude and/or vector (including magnitude and direction) of involuntary hand movement from the hand that is manipulating or handling the further device. The computer may transmit the generated data to a controller. The controller may then provide a counter force (of a magnitude and/or vector opposite to the involuntary hand movement) to the further device, e.g. on a tooltip or end effector of the device, to attenuate the involuntary hand movement from the hand to the further device, based on the data generated and transmitted by the computer.

The controller may further control a position (e.g. front, back, up, down, left or right position) and/or an orientation (e.g. an elevation angle, an azimuth angle and/or a torsion angle) of a positional sensor (of a plurality of positional sensors) relative to a respective sensor support and/or a position of the positional sensor relative to a circular support to which the positional sensor (and sensor support) is attached thereto (e.g. changing the position of the positional sensor, from a first position on the circular support to any other position along the circular support), based on the data generated and transmitted by the computer.

According to various embodiments, a control system of a monitoring system may include three components: a visual feedback loop for active guidance, a feedforward loop for tremor (e.g. of approximately 8-12 Hz) compensation and an open-loop control for tip actuation.

According to various embodiments, the visual feedback loop, for active guidance, may include a plurality of positional sensors (e.g. at least three positional sensors).

According to various embodiments, the visual feedback loop may further include any one or both of the magnification lens system and the image detector.

Further, according to various embodiments, the feedforward loop, for tremor (e.g. of approximately 8-12 Hz) compensation, may include the computer, the controller and the device and/or the further device. According to various embodiments, the device and/or further device may include respective at least one piezo actuator (or piezo driver). Further, according to various embodiments, the open loop control, for tip actuation, may include the device and/or the further device.

As an example, the visual feedback loop may include at least three positional sensors of the monitoring system 200a of the surgical system 200a, which may, in turn, provide a filtered and accurate three-dimensional tremor sensing (e.g. high-frequency tremor displacement and/or low-frequency drift error) in real time of a device (and/or a further device) and may further include an image detector mounted on a magnification lens system, which may, in turn, be calibrated and registered to the at least three positional sensors of the monitoring system 200a. Images may be obtained by the image detector (e.g. detected or determined) in real time (e.g. via the magnification lens system), for performing tip (e.g. tooltip) tracking and operation ground registration from an anatomy of a subject (e.g. person/patient). Operation ground registration may be used for target recognition and localization. A visual feedback control may be achieved with the operation ground registration and the obtained tip position from tip tracking.

A control law may then be applied to achieve active guidance in limiting the low-frequency drift error (e.g. approximate 8-12 Hz) of the device (and/or the further device). The high-frequency tremor displacement and the low-frequency drift error (detected by the positional sensors e.g. the at least three positional sensors) may be combined and transformed into the tip's (e.g. device tip) actuation coordinate system using an inverse kinematics model and may be fed into the open-loop actuation control system (i.e. open-loop control for tip actuation), while passing through a hysteresis model.

In addition, a monitoring system according to various embodiments of the present disclosure may have a low latency, which may be achieved (or provided) by way of using positional sensors (e.g. the plurality of positional sensors or any suitable optical sensors) which respectively has a very high sampling rate and transmitting rate. Accordingly, when data (e.g. of a device) is obtained by the positional sensors (i.e. with very high sampling rate and transmitting rate), a position and/or an orientation (e.g. three-dimensional position and/or three dimensional orientation) of a device may be readily (or almost immediately) computed for the purpose of providing tremor compensation via a controller. Accordingly, the process of data collection of a position and/or an orientation of a device to tremor compensation may be accomplished in real-time with high frequency.

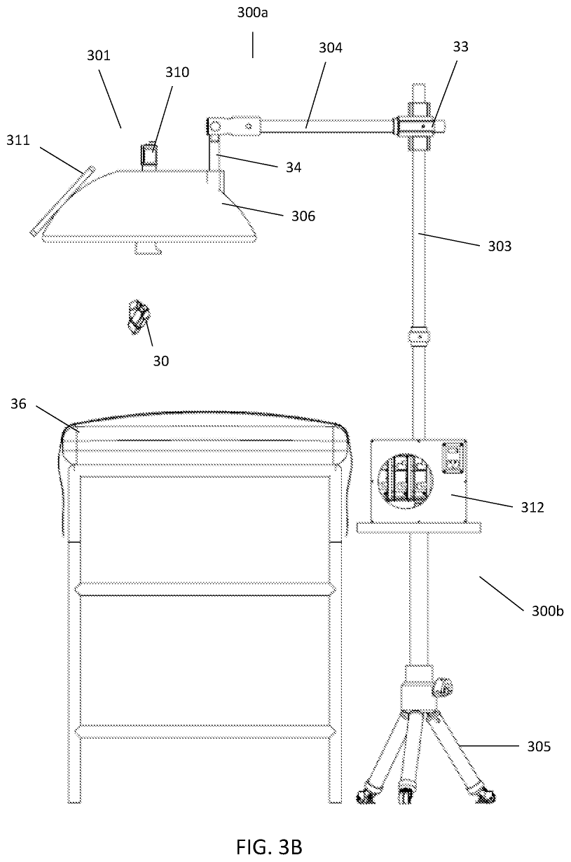

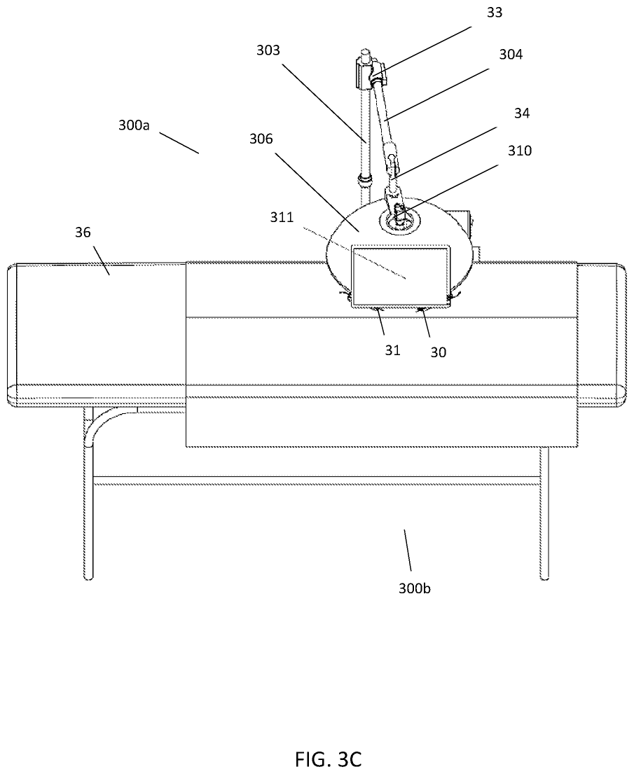

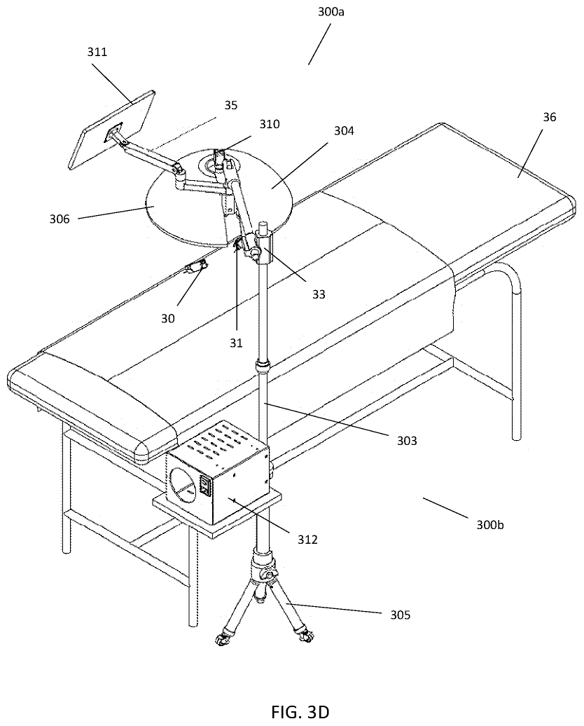

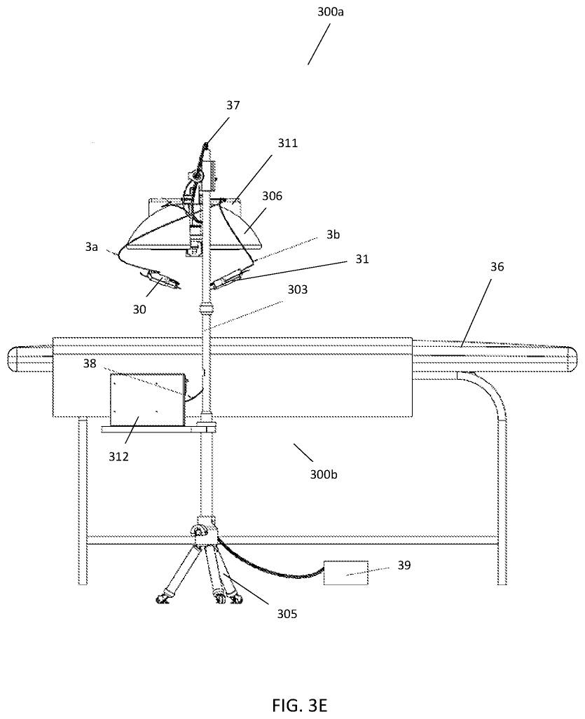

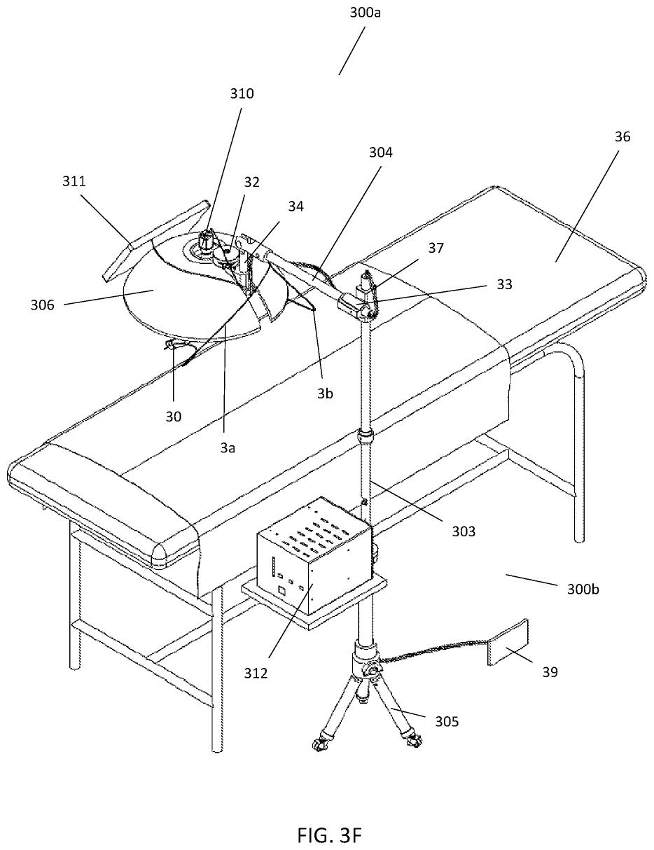

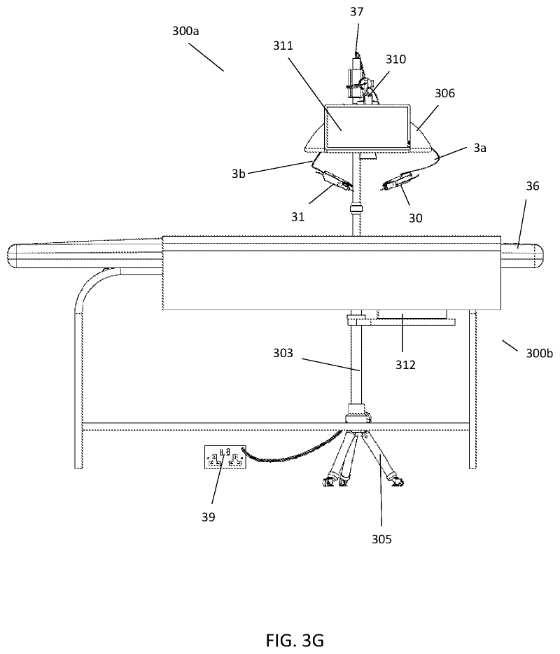

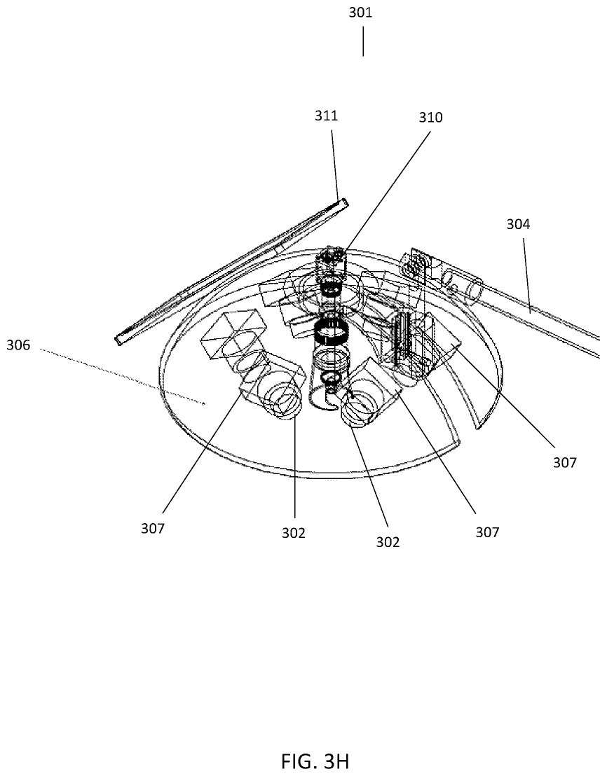

FIG. 3A shows a perspective view of the surgical system 300b according to various embodiments. FIG. 3B shows a front view of the surgical system 300b shown in FIG. 3A according to various embodiments. FIG. 3C shows a top view of the surgical system 300b shown in FIG. 3A according to various embodiments. FIG. 3D shows a magnified view of the surgical system 300b shown in FIG. 3A according to various embodiments. FIG. 3E shows a side view of the surgical system 300b shown in FIG. 3A according to various embodiments. FIG. 3F shows a perspective view of the surgical system 300b shown in FIG. 3A but with the controller arranged in a different orientation according to various embodiments. FIG. 3G shows another side view of the surgical system 300b shown in FIG. 3A according to various embodiments. FIG. 3H shows a magnified see-through view of a circular support of the monitoring system shown in FIG. 3A according to various embodiments.

FIGS. 3A-3G show a surgical system 330b including a monitoring system 300a and a device 30 according to various embodiments.

According to various embodiments, the surgical system 300b may further include a further device 31.

According to various embodiments, the monitoring system 300a may include a magnification lens system 301. As shown in FIGS. 3A-3H, the magnification lens system 301 is illustrated as a digital microscope.

As shown in FIGS. 3A-3H, the circular support 306 is illustrated as a concave structure (e.g. dome) with a circular shape, and a through-hole centered along a central axis of the circular support 306. As shown, the magnification lens system 301 is attached to the circular support 306, such that the magnification lens system 301 is held by the circular support 306, and the magnification lens system 301 is positioned along a longitudinal axis of the through-hole such that a portion of the magnification lens system 301 is positioned within the through-hole and is surrounded by the circular support 306.

As shown in FIG. 3H, according to various embodiments, the monitoring system 300a may further include a plurality of positional sensors 302 in an arrangement around or surrounding the magnification lens system 301. As shown in FIG. 3H, the plurality of positional sensors 302 may be attached to one side, for example, an underside (e.g. concave side), of the concave structure (e.g. dome) in a circular arrangement around (or surrounding) the magnification lens system 301.

According to various embodiments, the plurality of positional sensors 302 may be arranged in a circular arrangement, for example, around a geometric circle or circumference, such that each positional sensor of the plurality of positional sensors 302 is positioned a predetermined distance away from a neighboring positional sensor.

As another example, the plurality of positional sensors 302 may be equilaterally spaced apart from each other.

As a further example, each positional sensor of the plurality of positional sensors 302 may be arranged in a circular arrangement around the magnification lens system 301 such that each positional sensor of the plurality of positional sensors 302 is positioned a predetermined distance away from the magnification lens system 301. For example, a first positional sensor of the plurality of positional sensors 302 may be positioned at a first predetermined distance away from the magnification lens system 301, a second positional sensor of the plurality of positional sensors 302 may be positioned at a second predetermined distance away from the magnification lens system 301 etc.

It may also be envisioned that in various embodiments, the plurality of positional sensors 302 may be arranged in any other suitable arrangement around the magnification lens system 301.

For example, the plurality of positional sensors 302 may be arranged such that a first set of the plurality of positional sensors 302 lies in a first plane, the first set including at least one positional sensor; a second set of the plurality of positional sensors 302 lies in a second plane, the second set including at least another positional sensor. In addition, a third set of the plurality of positional sensors 302 may lie in a third plane, the third set including at least a further positional sensor etc. According to various embodiments, the planes may be horizontal planes, and the planes (i.e. first plane, second plane, third plane etc.) may be parallel to one other. In other words, the plurality of positional sensors 302 may be positioned in a multiple layer arrangement, wherein each layer of the multiple layer arrangement may include at least one positional sensor of the plurality of positional sensors 302.

Alternatively, according to various embodiments, the plurality of positional sensors 302 may lie in a single plane.

According to various embodiments, the monitoring system 300a may further include a stand 303. The stand 303 may have a first end portion and a second end portion. The monitoring system 300a may, similar to FIG. 2A, include an overhanging arm 304 attached, via coupler 33, to the first end portion of the stand 303, such that the overhanging arm 304 extends from the first end portion of the stand 303 in a manner that is substantially perpendicular to the first end portion of the stand 303. The monitoring system 300a may, similar to FIG. 2A, further include a base 305 attached to the second end portion of the stand 303. As shown in FIGS. 3A-3G, the base 305 is illustrated as a tripod. It may also be envisioned that in various embodiments, the base 305 may include any other suitable structure to support and hold the stand 303, including the overhanging arm 304 extending from the first end portion of the stand 303, in an upright position.

According to various embodiments, the monitoring system 300a, further include a circular support 306 connected to the overhanging arm 304. As shown in FIGS. 3A-3G, the circular support 306 is connected to the overhanging arm 304 via a linkage 34 that is pivotally connected to the overhanging arm 304. Accordingly, the circular support 306 may be movable relative to the overhanging arm 304, for example, pivotally movable via linkage 34. It may also be envisioned that in various embodiments, the linkage 34 may be any suitable linkage that may couple the circular support 306 to the overhanging arm 304 and allow various degrees of movement (e.g. having three or 6 degrees of freedom) of the circular support 306 relative to the overhanging arm 304. According to various embodiments, the circular support 306 may be configured to hold the plurality of positional sensors 302 in an arrangement around or surrounding the magnification lens system 301.

According to various embodiments, the surgical system 300b in FIGS. 3A-3G may further include an image detector (or camera) 310 configured to detect a magnified image (e.g. of the device 30 and/or the further device 31) generated by the magnification lens system 301 of the monitoring system 300a. According to various embodiments, the image detector (or camera) 310 may be incorporated within the magnification lens system 301. It may also be envisioned that in various embodiments, the image detector (or camera) 310 may be a separate component from the magnification lens system 301.

According to various embodiments, the surgical system 300b in FIGS. 3A-3G may further include a monitor 311 coupled to the image detector 310. According to various embodiments, the monitor may be configured to display the magnified image generated by the magnification lens system.

As shown in FIGS. 3A-3C, 3F-3G, the monitor 311 may be attached to an outer surface of the circular support 306. Alternatively, as shown in FIG. 3D, the monitor 311 may be coupled to overhanging arm 304 via linkages 35. Linkages 35 may be configured to allow the monitor 311 to be movable with (various degrees of freedom) relative to the overhanging arm 304, thereby allowing the monitor to move independently of the circular support. It may also be envisioned that in various embodiments, the monitor 311 may be positioned at any other suitable location, for example, coupled to any portion of the stand 303, or placed on a platform that may be coupled to a portion of the monitoring system 300a or the surgical system 300b, or placed on a platform that may be positioned at a predetermined distance away from the monitoring system 300a or the surgical system 300b.

According to various embodiments, the surgical system 300b in FIGS. 3A-3G may further include a computer (not shown) coupled to the plurality of positional sensors 302.

According to various embodiments, the surgical system 300b in FIGS. 3A-3H may further include a controller 312 coupled to the computer (not shown), the controller 312 may be coupled to the device 30 and/or the further device 31. According to various embodiments, the controller 312 may be further coupled to a plurality of positional sensors 302 including the coupling or couplings which connect the plurality of positional sensors 302 to the plurality of sensor supports 307 and to the circular support 306.

According to various embodiments, the computer (not shown) of surgical system 300b may be configured to receive data on a position (e.g. three-dimensional position) and/or an orientation (e.g. three-dimensional orientation) of the device 30 and/or the further device 31 as determined by the plurality of positional sensors 302. In other words, the plurality of positional sensors 302 may be configured to transmit data on the position (e.g. three-dimensional position) and/or the orientation (e.g. three-dimensional orientation) of the device 30 and/or the further device 31 to the computer (not shown).

According to various embodiments, the computer (not shown) of surgical system 300b may be configured to transmit data (i.e. data received from the plurality of positional sensors) on the position (e.g. three-dimensional position) and/or the orientation (e.g. three-dimensional orientation) of the device 30 and/or the further device 31 to the controller 312. In other words, the controller 312 may be configured to receive data on the position (e.g. three-dimensional position) and/or the orientation (e.g. three-dimensional orientation) of the device 30 and/or the further device 31 from the computer (not shown).

According to various embodiments, the controller 312 may be configured to control the device 30 and/or the further device 31 based on data (or feedback data, i.e. data received from the computer (not shown) and the plurality of positional sensors 302) on the position (e.g. three-dimensional position) and/or the orientation (e.g. three-dimensional orientation) of the device 30 and/or the further device 31.

According to various embodiments, the controller 312 may be further configured to control (e.g. move) a position (e.g. front, back, up, down, left or right position) and/or an orientation (e.g. an elevation angle, an azimuth angle and/or a torsion angle) of at least one positional sensor (of the plurality of positional sensors 302) relative to a respective sensor support and/or a position of the at least one positional sensor relative to the circular support 306 to which the at least one positional sensor (and respective at least one sensor support) is attached thereto (e.g. changing the position of the positional sensor, from a first position on the circular support 306 to any other position along the circular support 306), based on data (or feedback data, i.e. received from the computer (not shown) and the plurality of positional sensors 302) on the position (e.g. three-dimensional position) and/or the orientation (e.g. three-dimensional orientation) of the device 30 and/or the further device 31.

In other words, the controller 312 of surgical system 300b may be configured to control (e.g. move) the device 30 and/or the further device 31 and/or a positional sensor and/or the plurality of positional sensors 302, based on the data of the three-dimensional position and/or a three-dimensional orientation of the device 30 and/or the further device 31.

As shown in FIGS. 3E-3F, the monitoring system 300a may be electrically coupled to a socket box 39 on a wall. Wires may also be guided along and placed within (e.g. inside) stand 303 (e.g. when stand 303 is an annular structure with a cavity therein). As shown, the stand 303 may have at least one aperture (or opening, e.g. side hole) on a frame of the stand 303. Accordingly, as shown, a wire 37 for the image detector 310 (or camera) and the magnification lens system 301 may pass through a first aperture on the top portion of the frame of the stand 303 and a controller wire 38 may pass through another aperture on the middle portion of the frame of the stand 303.

According to various embodiments, a wire winder 32 may be provided. The wire winder 32 may be configured to automatically wind as well as unwind wires using (or exerting) only a small amount of force on the wire. According to various embodiments, the wire winder 32 may be configured to detect a small force exerted (e.g. by a user) on a wire and, upon detection of the small force exerted on the wire, the wire winder 32 may thereafter begin to wind or unwind the wire.

With reference to FIGS. 3C-3E and 3G, wires 3a and 3b for connecting to the device 30 and the further device 31, respectively, may be wound around the wire winder 32 before being connected to the device 30 and the further device 31. Accordingly, as an illustration, when a doctor uses a very small force to drag (or move) the device 30 and/or the further device 31 over a distance (e.g. long distance) from one location to another, the wire winder 32 is able to detect the small force exerted by the doctor on the wire 3a of the device 30 and/or on the wire 3b of the further device 31 and thereafter the wire winder 32 may begin to unwind the wire 3a and/or wire 3b, so that the doctor may only require to exert even less force than the original small force to drag (or move) the device 30 and/or the further device 31.

According to various embodiments, the monitoring system 300a, as shown in FIGS. 3A-3G, may be configured (or used) to monitor the device 30 and/or the further device 31 during surgery, for example, microsurgery, performed on a subject (patient) lying on operating table 36. According to various embodiments, the monitoring system 300a and the device 30 may form a surgical system 300b.

According to various embodiments, the device 30 and/or a further device 31 may be monitored when placed (or positioned) in a space below the plurality of positional sensors 302 which are attached to the circular support 306.

In other words, the plurality of positional sensors 302 may be attached to the circular support 306. Further, when attached to the circular support 306, a position (e.g. front, back, up, down, left or right position) and/or an orientation (e.g. an elevation angle, an azimuth angle and/or a torsion angle) of each positional sensor of the plurality of positional sensors 302 may be adjusted such that each positional sensor of the plurality of positional sensors 302 has a field of view or line of sight (or overlapping fields of views or lines of sights) in a direction downwards from the circular support 306 (or a line of slight to the device 30 and/or the further device 31). Accordingly, when the circular support 306 and the plurality of positional sensors attached thereto are overhanging (or overhead) the device 30 and/or a further device 31, the plurality of positional sensors 302 (e.g. at least three positional sensors) may be configured to monitor the device 30 and/or a further device 31 and determine a three-dimensional position and/or a three-dimensional orientation of the device 30 and/or a further device 31, based on a detection of the device 30 and/or the further device 31 (e.g. detection of all of at least three light sources of the device 30 and/or at least three light sources of the further device 31) by at least two positional sensors of the plurality of positional sensors 302 (e.g. at least three positional sensors).

According to various embodiments, the device 30 and/or a further device 31 may be a surgical tool (e.g. left handheld surgical tool) and/or a further surgical tool (e.g. right handheld surgical tool) which may be manipulated by hand (of a surgeon) for operation on the subject (i.e. patient).

According to various embodiments, each of or either the device 30 and/or the further device 31 may be a holder, a single-blade cutter (e.g. knife), a dual-blade cutter (e.g. scissors), a fluid injector (e.g. syringe) or any other suitable surgical tool.

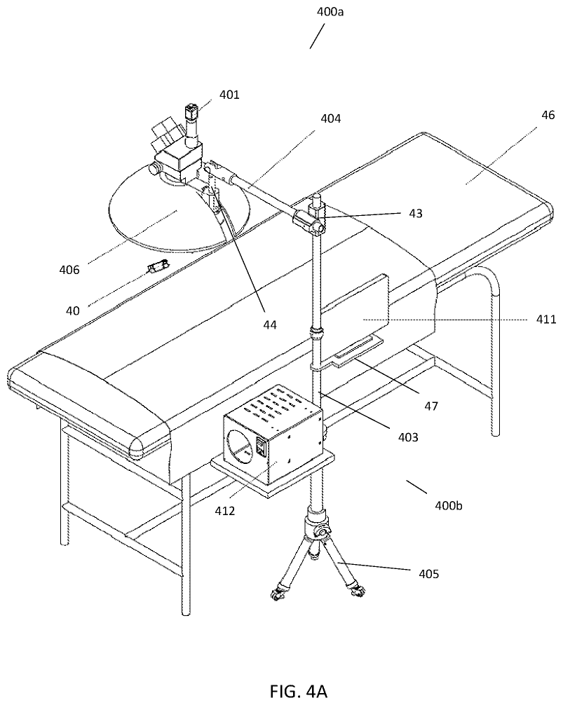

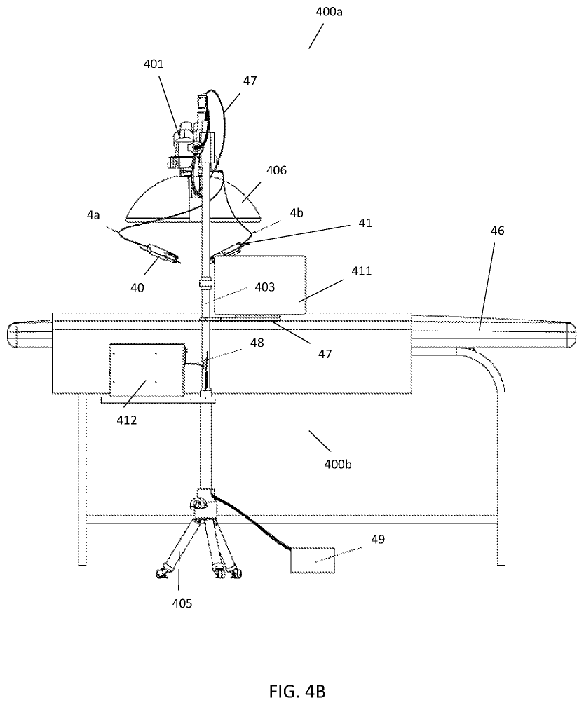

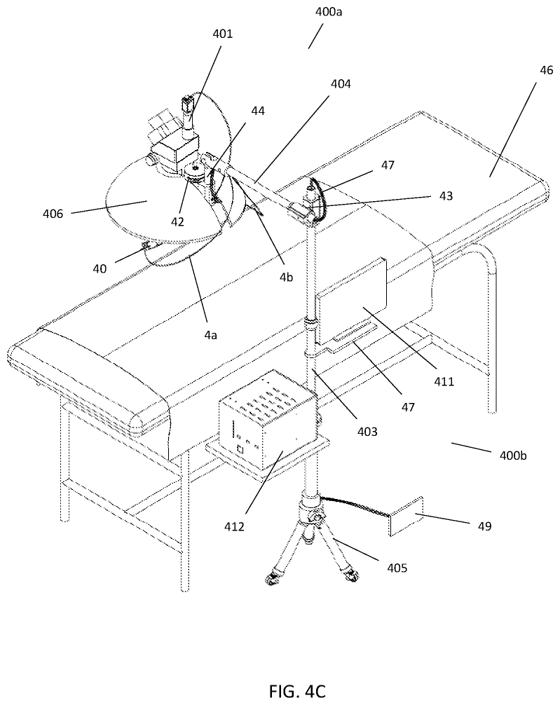

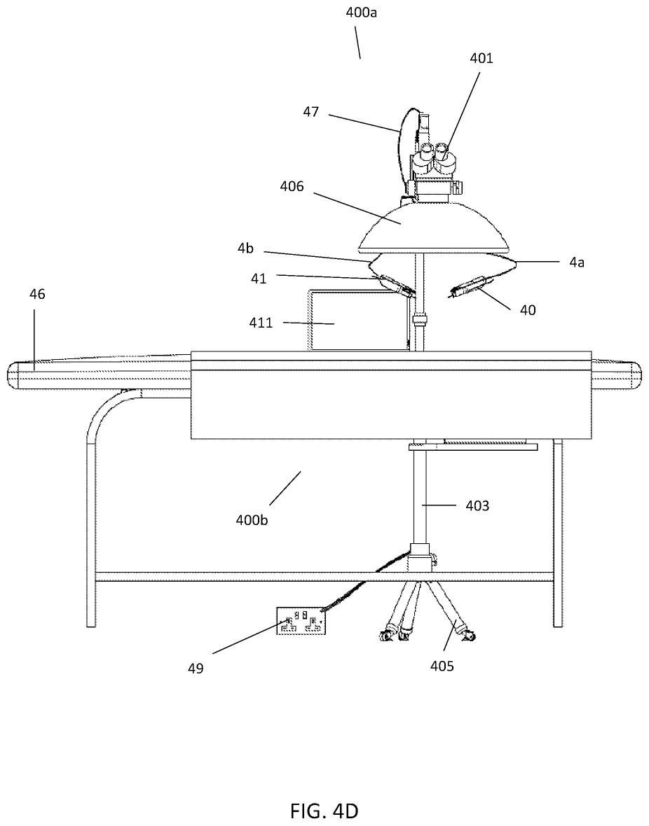

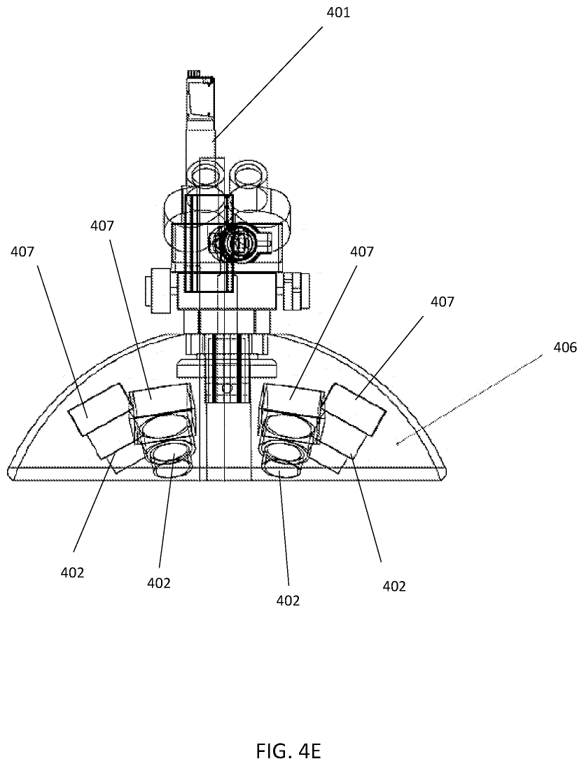

FIG. 4A shows a perspective view of the surgical system 400b according to various embodiments. FIG. 4B shows a side view of the surgical system 400b shown in FIG. 4A according to various embodiments. FIG. 4C shows a perspective view of the surgical system 400b shown in FIG. 4A but with the stand arranged in a different orientation according to various embodiments. FIG. 4D shows another side view of the surgical system 400b shown in FIG. 4A according to various embodiments. FIG. 4E shows a see-through view of a circular support 406 of the monitoring system shown in FIG. 4A according to various embodiments.

The embodiments in FIGS. 4A-4E may be similar or identical to the embodiments in FIGS. 3A-3H, except that the digital microscope 301 is replaced by an optical microscope 401, and further, a platform 47 may be provided (or coupled) on a portion of the stand 403 to support or hold a monitor 411.

FIGS. 4A-4D show a surgical system 400b including a monitoring system 400a and a device 40. The surgical system 400b may further include a further device 41.

As shown, according to various embodiments, the monitoring system 400a may include a stand 403 having a first end portion and a second end portion. An overhanging army 404 may be attached to the first end portion of the stand 403 via coupler 43, and a base 405 may be attached to the second end portion of the stand.

According to various embodiments, the monitoring system 400a may further include a circular support 406 for holding a plurality of positional sensors 402 via a plurality of sensor supports 407 (shown in FIG. 4E). The circular support 406 may be coupled to the overhanging arm 404 via linkage 44.

According to various embodiments, the monitoring system 400a may further include the digital microscope 401 which may be attached to the circular support 406.

According to various embodiments, the surgical system 400b may further include a monitor 411 which may be positioned on platform 47.

According to various embodiments, the surgical system 400b may further include a controller 412 which may be configured to control (e.g. move) the device 40 and/or the further device 41 and/or a positional sensor and/or the plurality of positional sensors 402, based on a data of the three-dimensional position and/or a three-dimensional orientation of the device 40 and/or the further device 41.

According to various embodiments, the monitoring system 400a may be electrically coupled to a socket box 49 on a wall. Wires may also be guided along and placed within (e.g. inside) stand 403 (e.g. when stand 403 is an annular structure with a cavity therein). As shown, the stand 403 may have at least one aperture (or opening, e.g. side hole) on a frame of the stand 403. Accordingly, as shown, a wire 47 for the optical Microscope 401 may pass through a first aperture on the top portion of the frame of the stand 403 and a controller wire 48 may pass through another aperture on the middle portion of the frame of the stand 403.

According to various embodiments, a wire winder 42 may be provided. The wire winder 42 may be configured to automatically wind and unwind wires using (or exerting) only a small amount of force on the wire. Thus, wires 4a and 4b for connecting to the device 40 and the further device 41, respectively, may be wound around the wire winder 42 before being connected to the device 40 and the further device 41.

According to various embodiments, the monitoring system 400a, as shown in FIGS. 4A-4D, may be configured (or used) to monitor the device 40 and/or the further device 41 during surgery, for example, microsurgery, performed on a subject (patient) lying on operating table 46. According to various embodiments, the monitoring system 400a and the device 40 may form a surgical system 400b.

FIG. 5A shows a perspective view of a portion of a monitoring system including the circular support 506, the plurality of sensor supports 507, the magnification lens system 501 and a plurality of recesses 51 according to various embodiments. FIG. 5B shows a perspective view of a portion of a monitoring system including the circular support 506, the plurality of sensor supports 506, the magnification lens system 501 and a plurality of carriages 54 according to various embodiments.

FIGS. 5A-5B show a circular support 506, a plurality of sensor supports 507 attached to the circular support 506, a plurality of positional sensors 502 attached to the plurality of sensor supports 507, a magnification lens system 501, all of which may respectively correspond to the circular support, plurality of sensor supports, plurality of positional sensors, plurality of positional sensors and magnification lens system in FIGS. 1, 2, 3A-3H and 4A-4E, according to various embodiments.

In FIGS. 5A-5B, the circular support 506 is illustrated as an annular rail.

According to various embodiments, the magnification lens system 501 and the plurality of positional sensors 502 may be coupled to the circular support 506 (i.e. annular rail). Accordingly, the magnification lens system 501 and the plurality of positional sensors 502 may move in tandem with the circular support 506.

Alternatively, according to various embodiments, the magnification lens system 501 and the plurality of positional sensors 502 may move independently of one another.

Various embodiments of the attachment of the plurality of sensor supports to the circular support, the circular support itself and the plurality of sensor supports themselves will now be illustrated below with reference to FIGS. 5A-5B.

According to various embodiments, the plurality of sensor supports 507 may be attached to the circular support 506 (i.e. annular rail) by any suitable means.

In FIG. 5A, the circular support 506 (i.e. annular rail) may include a plurality of recesses 51 (e.g. through-holes) located at predetermined portions of a wall of the circular support 506. Each recess of the plurality of recesses 51 may be configured to receive a coupler that is, in turn, configured to attach a respective sensor support to the circular support 506.

According to various embodiments, the plurality of recesses 51 may include a number (e.g. one or more than one) of groups of recesses, wherein each group of recesses may include any number of recesses including one recess or more than one recess. Each group of recesses may be configured to receive a corresponding number of couplers for attachment of one sensor support to the circular support 506 (i.e. to the portion of the circular support 506 where the group of recesses in question is located). As shown in FIG. 5A, the circular support 506 may include six groups of recesses (each group of recesses including two recesses) for attachment of six sensor supports (and six positional sensors) thereto. As shown in FIG. 5A, three sensor supports (and three positional sensors) are attached to a first semi-circular rail component of the circular support 506 and another three sensor supports (and three positional sensors) are attached to a second semi-circular rail component of the circular support 506. It may also be envisioned that in various embodiments, any number of sensor supports (and any number of positional sensors) may be attached to the circular support 506, and accordingly, any number of recesses may be provided to cater to any number (e.g. desired number) of sensor supports.

According to various embodiments, a plurality of groups of recesses may be positioned equilaterally around the circular support 506 (i.e. annular rail) such that the plurality of sensor supports 507 (and positional sensors) may be equilaterally spaced apart from each other, surrounding magnification lens system 501, while the plurality of sensor supports 507 (and positional sensors) are attached to the circular support 506. It may also be envisioned that in various embodiments, a plurality of groups of recesses (e.g. at least three groups of recesses) may be positioned at any portion of the wall of the circular support 506. Accordingly, at least three sensor supports (and at least three positional sensors) may be positioned at (and attached to) any portion of the wall of the circular support 506 (e.g. on an inner surface of the wall).

According to various embodiments, each sensor support of the plurality of sensor supports 507 may be interchangeably attached to each predetermined portion of a wall of the circular support 506 where a group of recesses is located. Accordingly, a number of groups of recesses on any portion of the wall of the circular support 506 may be more than a number of sensor supports or more than a number of positional sensors, such that each sensor support and/or each positional sensor may be interchangeably attached to any portion of the wall of the circular support 506 where a group of recesses is located (and available).

In FIG. 5A, according to various embodiments, the plurality of sensor supports 507 may be attached to an inner surface of a wall of an annular rail and held in a fixed position by a coupler, such that the plurality of sensor supports 507 are immovable relative to the circular support 506.

In FIG. 5B, according to various embodiments, the circular support 506 (i.e. annular rail) may include a plurality of carriages slidably coupled or attached to the circular support 506 (i.e. annular rail). Each carriage 54 may, in turn, be attached to one sensor support of the plurality of sensor supports 507. In other words, each carriage 54 may be slidably coupled or attached to the circular support 506 on one side of the carriage 54, and the carriage 54 may be attached to one sensor support on another side of the carriage 54.

As shown in FIG. 5B, the circular support 506 (i.e. annular rail) may include a plurality of segments along an inner surface of a wall of the circular support 506. Each segment 53 may be a predetermined portion of the circular support 506, and may be defined as a strip of rail of the circular support 506 (i.e. annular rail) between two stops. One stop may be located at a first end of the strip and another stop may be located at a second end of the strip.

According to various embodiments, each carriage 54 of the plurality of carriages may be interchangeably attached and slidably coupled or attached to each segment 53 of the plurality of segments of the circular support 506, respectively. According to various embodiments, each positional sensor of the plurality of positional sensors 502 may be interchangeably attached to each carriage 54 of the plurality of carriages. Accordingly, a number of segments of the circular support 506 (and/or a corresponding number of carriages) may be more than a number of sensor supports or more than a number of positional sensors, such that each sensor support and/or each positional sensor may be interchangeably attached to any portion of the wall of the circular support 506 where a segment 53 (and a corresponding carriage) is located (and available).

Alternatively, the circular support 506 (i.e. annular rail) may include a single continuous segment (not shown) along an inner surface of the wall of the circular support 506. According to various embodiments, a number of carriages on the single continuous segment may be more than a number of sensor supports or more than a number of positional sensors, such that each sensor support and/or each positional sensor may be interchangeably attached to any portion of the wall of the circular support 506 where a carriage is located (and available).

According to various embodiments, where the circular support 506 includes a plurality of segments, each carriage 54 may be configured to slide along a respective segment of the circular support 506 between the two stops of the respective segment. Alternatively, where the circular support 506 includes a single continuous segment, each carriage 54 may be configured to slide along the single continuous segment.

According to various embodiments, each carriage 54 may include a motor configured to move the carriage individually, for example, along a respective segment of the circular support 506 or along a single continuous segment of the circular support 506.

As shown in FIG. 5B, the circular support 506 may include a grating scale, on an inner surface of the wall of the circular support 506, which may detect (or determine) and provide information on a position of each sensor support (and corresponding positional sensor) on the annular rail (i.e. circular support 506) relative to the circular support 506 and may further be configured to provide information of the position of each sensor support of the plurality of sensor supports 507 relative to the circular support 506.

Various embodiments of the attachment of the plurality of positional sensors 502 to the plurality of sensor supports 507 will now be illustrated below with reference to FIGS. 5A-5B.

According to various embodiments, each sensor support of the plurality of sensor supports 507 may be attached to one positional sensor of the plurality of positional sensors 502 to hold the positional sensor. In other words, each sensor support of the plurality of sensor supports 507 may be configured to hold one positional sensor of the plurality of positional sensors 502 via a coupling between the sensor support and the positional sensor.

According to various embodiments, each positional sensor of the plurality of positional sensors 502 may be interchangeably attached to each sensor support of the plurality of sensor supports 507.

As shown in FIG. 5A, the coupling between each sensor support and each positional sensor may include a mechanical fastener. For example, the mechanical fastener may include a nail, a screw (e.g. a threaded screw), a bolt (e.g. bolt and nut), a stud, a rivet, or other suitable types of mechanical fastener. According to various embodiments, the coupling between each sensor support and each positional sensor of the plurality of positional sensors 502 may be manipulated to allow a position (e.g. front, back, up, down, left or right position) and/or an orientation (e.g. an elevation angle, an azimuth angle and/or a torsion angle) of the positional sensor to be adjusted relative to a respective sensor support. Accordingly, a position and/or an orientation of a positional sensor may be adjusted (e.g. manually), and once a desired orientation and/or position of the positional sensor is determined, the positional sensor may be attached to a respective sensor support and held firmly (or rigidly) in place via the coupling. For example, the coupling may be loosened (e.g. to loosely couple a positional sensor to a respective sensor support) to allow a position and/or an orientation of a positional sensor to be adjusted. The coupling may alternatively be tightened to firmly secure a positional sensor to a respective sensor support (such that the positional sensor is immovable relative to the sensor support).

According to various embodiments, the coupling (i.e. second coupling) between the sensor support and the positional sensor may include a flexible structure including a shape-retaining material. For example, the coupling may be a wire (e.g. metal wire, alloy wire, nitinol wire etc.). Accordingly, when the coupling includes a flexible structure including a shape-retaining material, a position (e.g. front, back, up, down, left or right position) and/or an orientation (e.g. an elevation angle, an azimuth angle and/or a torsion angle) of the positional sensor relative to a respective sensor support may be adjusted (e.g. by manual manipulation) and held in place (e.g. when no adjustment or manipulation is made) by way of, at least, manipulation of the flexible structure including a shape-retaining material.

As shown in FIG. 5B, the coupling between each sensor support and each respective positional sensor may include mechanical fasteners and/or at least one motor configured to move and adjust a position (e.g. front, back, up, down, left or right position) and/or an orientation (e.g. an elevation angle, an azimuth angle and/or a torsion angle) of the positional sensor relative to a respective sensor support.

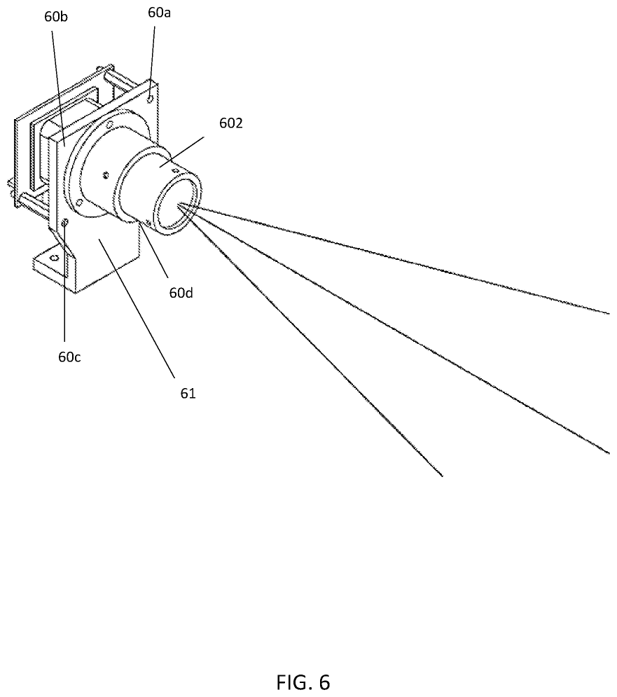

Various embodiments of the plurality of positional sensors will now be illustrated below with reference to FIG. 6.

FIG. 6 shows a perspective view of a positional sensor 602 according to various embodiments. The positional sensor 602 (e.g. PSD), may correspond to each positional sensor of the plurality of positional sensors in FIGS. 1, 2, 3A-3H, 4A-4E and 5A-5B.

According to various embodiments, the positional sensor 602 may define a working space for monitoring a device. A working space may be defined as a field of view of one positional sensor of the plurality of positional sensors. By way of example, when an omnidirectional light source is within a field of view of a positional sensor, the positional sensor may detect or receive a light emitted from the omnidirectional light source.

According to various embodiments, in order to enable a user to conveniently observe or determine the working space of each positional sensor of a plurality of positional sensors, at least one light producing element (e.g. laser source, light beam source etc.) may be provided on (e.g. attached/mounted to) a surface of each positional sensor, wherein the at least one light producing element may be configured to provide a visual indication or visual guide (e.g. approximate location or approximate range), on any suitable surface, of the working space of the positional sensor (i.e. the positional sensor in question). According to various embodiments, the at least one light producing element may produce a laser beam or a light beam (e.g. rigid light beam) that moves in tandem with a movement of the positional sensor, for example, movement of a position (e.g. front, back, up, down, left or right position) and/or an orientation (e.g. an elevation angle, an azimuth angle and/or a torsion angle) of the positional sensor relative to a respective sensor support and/or a position of the positional sensor relative to the circular support (e.g. changing the position of the positional sensor, from a first position on the circular support to any other position along the circular support).

For example, according to various embodiments, one light producing element (e.g. laser source, light beam source etc.) may be mounted on a surface of the positional sensor 602, for example, on (e.g. at an edge of) sensor plane 61 of the positional sensor 602, beside a lens of the positional sensor 602. The one light producing element may produce a laser beam or a light beam that is perpendicular (i.e. normal) to the sensor plane 61 on which the light producing element is mounted on or that is parallel to a longitudinal axis (or principal axis) of the lens of the positional sensor.

Alternatively, according to various embodiments, a plurality of light producing elements may be mounted on the surface of the positional sensor 602 (e.g. on the sensor plane 61). For example, three or four light producing elements may be equilaterally spaced around the lens of the positional sensor. As another example, each of four light producing elements may be mounted on the four corners 60a, 60b, 60c and 60d of the sensor plane 61 of the positional sensor 602, respectively.

According to various embodiments, each of the plurality of light producing elements may be configured to respectively produce a laser beam or a light beam.

According to various embodiments, each laser beam or light beam of each of the plurality of light producing elements may be configured to converge (e.g. meet) together on a point (on a surface or in space) along a longitudinal axis (or principal axis) of the lens of the positional sensor. Accordingly, when a plurality of laser beams or light beams of a plurality of light producing elements are set to converge toward a predetermined point along a longitudinal axis (or principal axis) of the lens of the positional sensor 602, the predetermined point where the laser beams or light beams converge may indicate an approximate location (e.g. center) of the working space of the positional sensor 602 at a predetermined distance away from the sensor plane 61 of the positional sensor 602. For example, when a surface (e.g. of a work base) is brought within the working space of the positional center at the predetermined distance from the sensor plane 61 where the plurality of laser beams or light beams of the plurality of light producing elements converge, a reflection of the plurality of laser beams or light beams of the plurality of light producing elements on the surface (i.e. of a work base) would appear as a single dot.

Alternatively, according to various embodiments, each laser beam or light beam of each of the plurality of light producing elements may be configured (or set) to indicate (e.g. on a surface within a working space of the positional sensor) an approximate location of an edge of the working space of the positional sensor 602, along any suitable range or distance away from the sensor plane 61 of the positional sensor. For example, when a surface (e.g. of a work base) is brought within the working space of the positional center within a suitable range or distance from the sensor plane 61, a reflection of the laser beam or light beam of each of the plurality of light producing elements on the surface would indicate an approximate location of an edge of the working space of the positional sensor.

According to various embodiments, an overlap of at least two working spaces of at least two respective positional sensors (e.g. of at least three positional sensors of a monitoring system) would define an effective working space of the monitoring system for monitoring a device. In other words, an effective working space may be defined as the overlap of at least two fields of views of respective at least two positional sensors (e.g. of at least three positional sensors of a monitoring system). As an example, when a device (e.g. a surgical tool) including at least three light sources (e.g. on a handle of the device) is within an effective working space of the monitoring system, the at least two positional sensors of the monitoring system (which defines the effective working space) may determine a three-dimensional orientation position (e.g. front, back, up, down, left or right position) and/or a three-dimensional orientation of the device. In other words, when at least two positional sensors of the plurality of positional sensors of the monitoring system detect (or receive) a light emitted from each of at least three light sources of the device, the at least two positional sensors may determine a three-dimensional position and/or a three-dimensional orientation of the device.

Accordingly, by providing (e.g. attaching/mounting) at least one light producing element (e.g. laser source, light beam source etc.) on a surface of each positional sensor of a plurality of positional sensors, an effective working space of the plurality of positional sensors may be readily identified or calibrated (e.g. by a user) by adjustment of a position (e.g. front, back, up, down, left or right position) and/or an orientation (e.g. an elevation angle, an azimuth angle and/or a torsion angle) of at least one positional sensor relative to respective at least one sensor support and/or a position of at least one positional sensor relative to the circular support (e.g. changing the position of at least one positional sensor, from a first position on the circular support to any other position along the circular support) such that a light source or light beam produced by at least one light producing element of respective at least two positional sensors of the plurality of positional sensors overlap.

According to various embodiments, a working space of each positional sensor of a plurality of positional sensors as well as an effective working space of a plurality of positional sensors may be calibrated manually (e.g. by a user) or automatically (e.g. by motorized means), as illustrated below with reference to FIGS. 5A-5B.

In FIGS. 5A-5B, each of the plurality of positional sensors 502 may be provided with at least one light producing element (e.g. laser source, light beam source etc.) on (e.g. attached/mounted to) a surface of each positional sensor, wherein the at least one light producing element may be configured to provide a visual indication or visual guide (e.g. approximate location or approximate range), on any suitable surface, of the working space of the positional sensor (i.e. the positional sensor in question).

According to various embodiments, the at least one light producing element may produce a laser beam or a light beam (e.g. rigid light beam) that moves in tandem with a movement of the positional sensor.

With reference to FIG. 5A, according to various embodiments, a working space of each positional sensor of a plurality of positional sensors 502 may be calibrated by way of a manual calibration system (i.e. manually). For example, for each positional sensor of a plurality of positional sensors 502, a position (e.g. front, back, up, down, left or right position) and/or an orientation (e.g. an elevation angle, an azimuth angle and/or a torsion angle) of each positional sensor relative to a respective sensor support of the plurality of sensor supports 507 and/or a position of each positional sensor relative to the circular support 506 may be adjusted manually by manipulation of the coupling between positional sensor and respective sensor support to which the positional sensor is attached. Accordingly, as shown in FIG. 5A, a position (e.g. front, back, up, down, left or right position) and/or an orientation of each positional sensor on the circular support 506 (i.e. on the annular rail or on each semi-circular rail component of the circular support 506) and/or a position of each positional sensor relative to the circular support 506 and, in turn, a working space of each positional sensor of the circular support 506, may be adjusted manually by manipulating mechanical fasteners (e.g. nails and screws).

Accordingly, an effective working space of the plurality of positional sensors 502 may be calibrated (e.g. created or set) by manual adjustment of a position (e.g. front, back, up, down, left or right position) and/or an orientation (e.g. an elevation angle, an azimuth angle and/or a torsion angle) of at least one positional sensor (i.e. of the plurality of positional sensors 502) relative to respective at least one sensor support and/or a position of at least one positional sensor relative to the circular support 506 (i.e. annular rail) (e.g. changing the position of at least one positional sensor, from a first position on the circular support 506, i.e. annular rail, to any other position along the circular support 506) such that a light source or light beam produced by at least one light producing element of respective at least two positional sensors of the plurality of positional sensors 502 overlap.

With reference to FIG. 5B, according to various embodiments, a working space of each positional sensor of a plurality of positional sensors 502 as well as an effective working space of a plurality of positional sensors 502 may be calibrated by way of an automatic calibration system (i.e. automatically). For example, each carriage 54 may include a motor configured to move the carriage 54, including a sensor support and a positional sensor, both of which are attached to the carriage 54, along the circular support 506 (i.e. annular rail). Further, a coupling between each sensor support and each positional sensor (on a carriage 54) may include at least one motor configured to move and adjust a position (e.g. front, back, up, down, left or right position) and/or an orientation (e.g. an elevation angle, an azimuth angle and/or a torsion angle) of the positional sensor relative to the sensor support. Accordingly, as shown in FIG. 5B, according to various embodiments, a position of each positional sensor relative to the circular support 506 and, in turn, a working space of each positional sensor of the circular support 506, may be adjusted automatically by movement of a respective carriage 54 (i.e. to which the positional sensor and sensor support are attached thereto) effected by a motor. Further, according to various embodiments, a position (e.g. front, back, up, down, left or right position) and/or an orientation (e.g. an elevation angle, an azimuth angle and/or a torsion angle) of each positional sensor relative to a respective sensor support and/or a position of each positional sensor relative to the circular support 506 and, in turn, a working space of each positional sensor of the circular support 506, may be adjusted automatically by way of another at least one motor included in the positional sensor.

According to various embodiments, an effective working space of the plurality of positional sensors 502 may be calibrated (e.g. created or set) by way of at least one motor configured to adjust a position (e.g. front, back, up, down, left or right position) and/or an orientation (e.g. an elevation angle, an azimuth angle and/or a torsion angle) of at least one positional sensor relative to respective at least one sensor support and/or a position of at least one positional sensor relative to the circular support 506 (i.e. annular rail) (e.g. changing the position of at least one positional sensor, from a first position on the circular support 506, i.e. annular rail, to any other position along the circular support 506) such that a working space of respective at least two positional sensors of the plurality of positional sensors 502 overlap.

According to various embodiments, each positional sensor of the plurality of positional sensors 502 may be configured to track (e.g. using any suitable sensor) a device (and/or a further device) including at least three light sources and move (e.g. by way of a motor included in the carriage 54 and the at least one motor included in the coupling between each positional sensor and each respective sensor support), such that a location (or position) of a working space of each positional sensor corresponds to (or coincides/overlaps with) a location of the device (and/or a further device). In other words, each positional sensor of the plurality of positional sensors 502 may be “self-adjusting”, in the manner that each positional sensor may be configured to, when in operation, follow a location (or position) of the device (and/or a further device) to ensure that the device (and/or a further device) is always or constantly within an area (e.g. in the center) defining the working space of the positional sensor.

Accordingly, according to various embodiments, at least two positional sensors of the plurality of positional sensors 502 may be configured to track (e.g. using any suitable sensor) a device (and/or a further device) including at least three light sources and move (e.g. by way of a motor comprised in each respective carriage 54 and the at least one motor comprised in the coupling between each respective positional sensor and each respective sensor support), such that a location (or position) of a working space of each of the at least two positional sensors corresponds to (or coincides/overlaps with) a location of the device (and/or a further device). In this manner, according to various embodiments, where there are at least three positional sensors, at least two positional sensors (e.g. of any pair of the at least three positional sensors) may be configured to, when in operation, created or set an effective working space that corresponds to (or coincides/overlaps with) a location of the device (and/or a further device).

Accordingly, according to various embodiments, the working space, as well as an effective working space, of a monitoring system is flexible (e.g. movable) and changeable (e.g. change in position and/or enlarge or reduce in size). Accordingly, according to various embodiments, a layout and/or a configuration of a plurality of positional sensors (e.g. at least three positional sensors) attached to a circular support may provide a flexible (e.g. movable) and changeable (e.g. change in position and/or enlarge or reduce in size) workspace (e.g. location for placing a device for monitoring of the device) for monitoring a device, while ensuring that at least three light sources on a device (e.g. three light sources on a handle of the device) is detected by at least two positional sensors of at least three positional sensors of a monitoring system at any reasonable pose (e.g. position, location and/or orientation) of the device.

As shown in FIGS. 5A-5B, six PSDs may be provided and attached to a circular support (e.g. three PSDs on a left semi-circular rail of the circular support and three PSDs on a right semi-circular rail of the circular support), the six PSDs configured to detect and monitor a position and/or a movement of a device (e.g. a portable left handheld device) and/or a further device (e.g. a portable right handheld device) within a wide range of different or varying poses (e.g. position and/or orientation) of the device, while providing a large workspace for detecting and monitoring the device. In the example of FIGS. 5A-5B, three PSDs may be configured to detect the portable left handheld device from a left hand motion and another three PSDs may be configured to detect the portable right handheld device from a right hand motion. It may also be envisioned that in various embodiments, more than six PSDs may be employed and provided, either on a single plane (e.g. single layer) or on multiple planes (e.g. multiple layer), to increase the size of the workspace for detecting and monitoring a device and/or a further device. It may also be envisioned that in various embodiments, at least three PSDs may be employed and provided. Accordingly, a monitoring system may be configured with multiple PSDs (e.g. at least three PSDs) with different arrangement in space (e.g. with the multiple PSDs attached to a circular support in space).

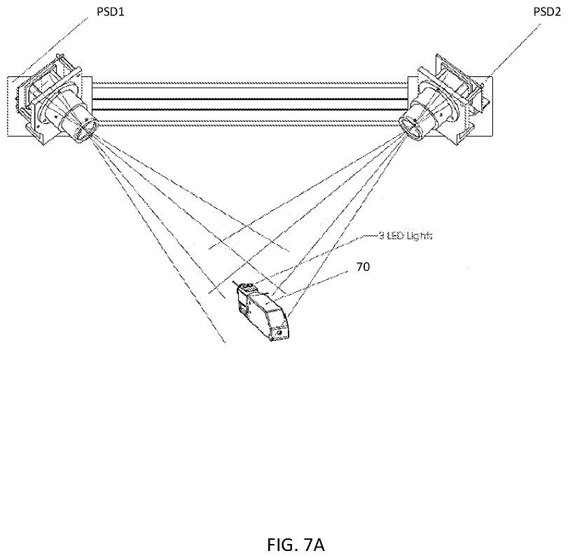

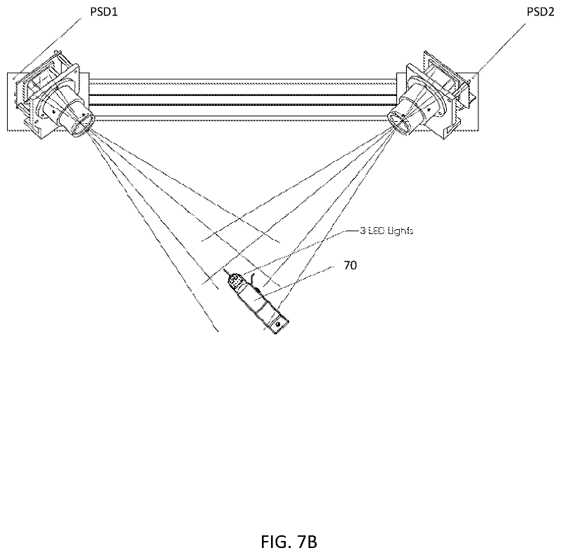

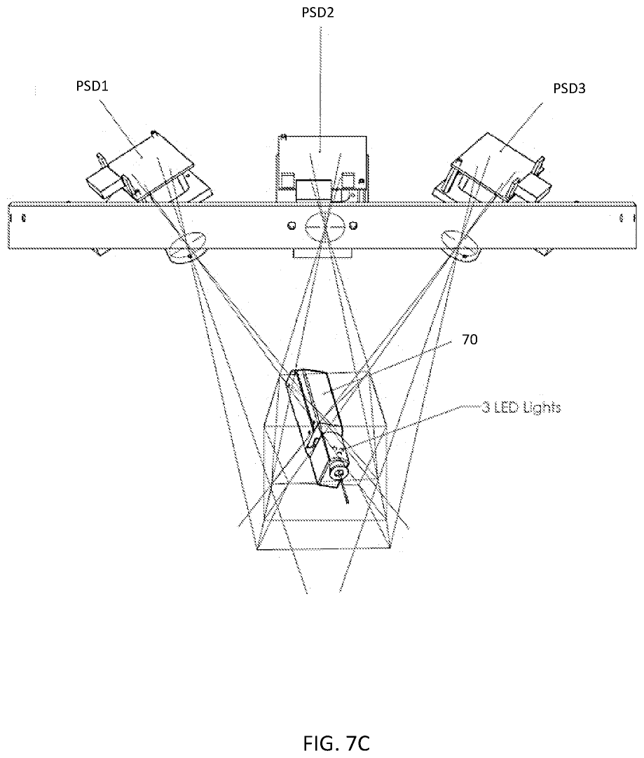

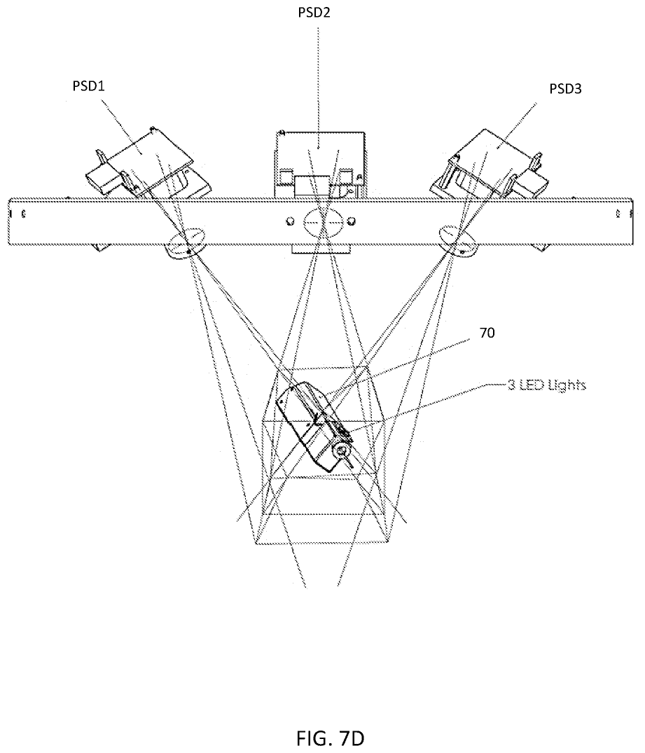

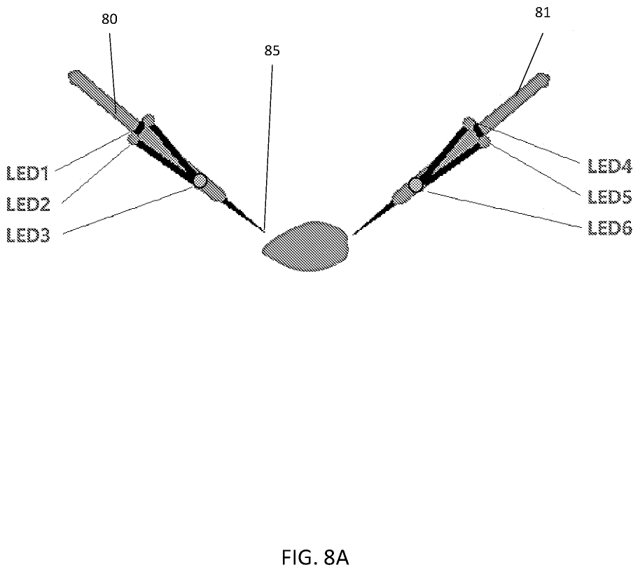

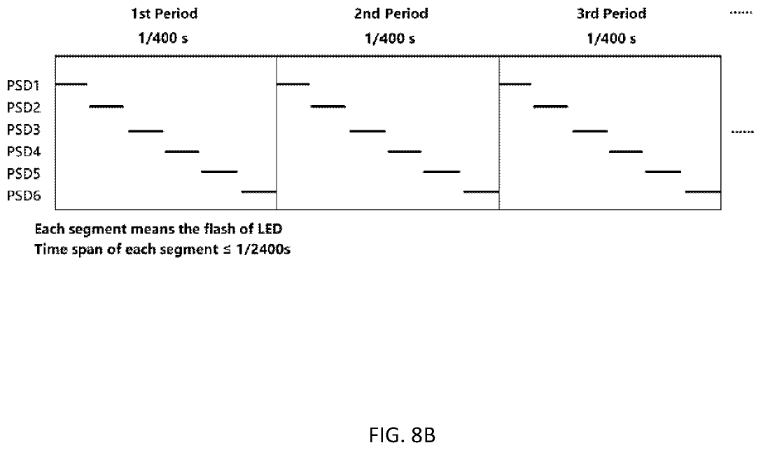

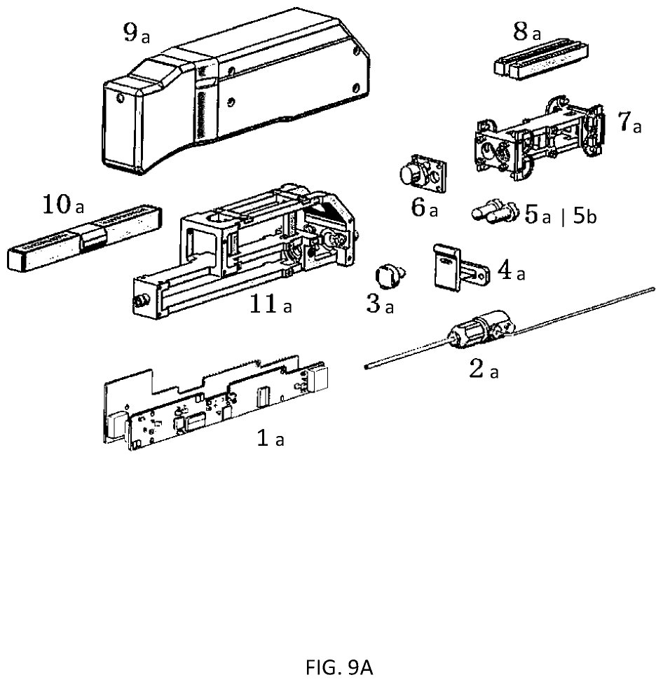

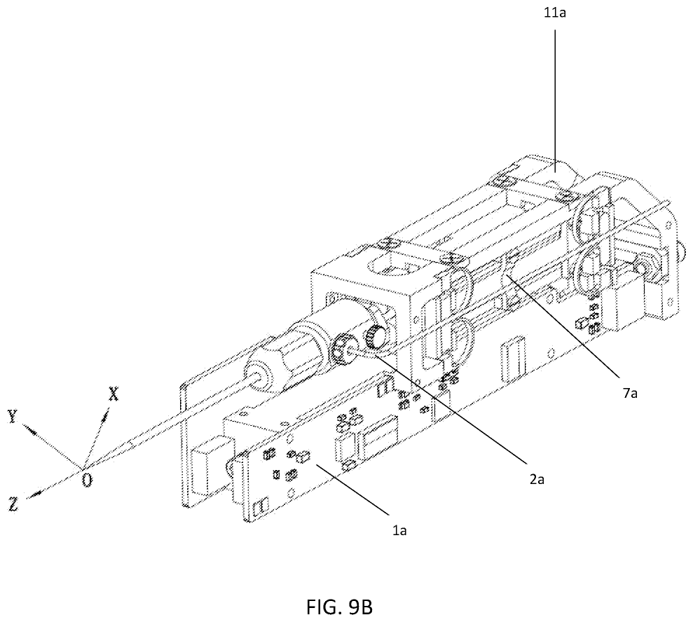

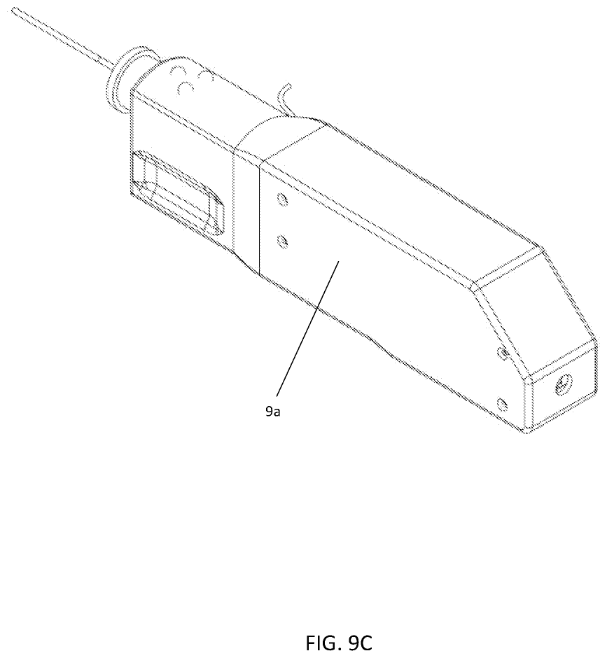

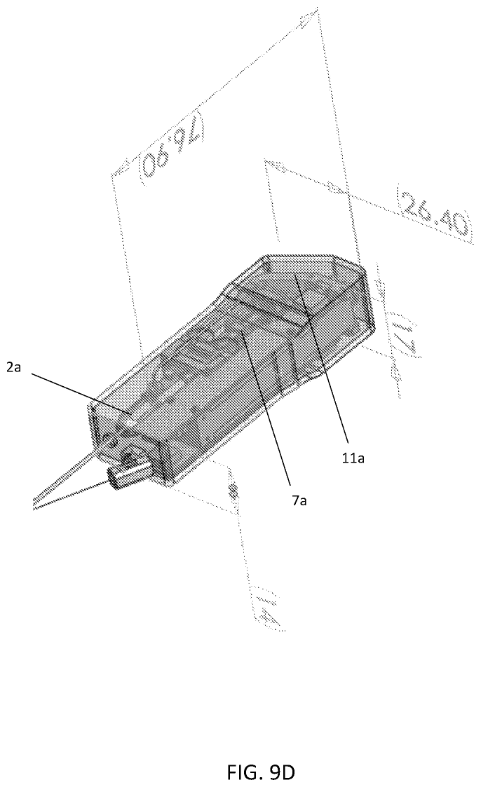

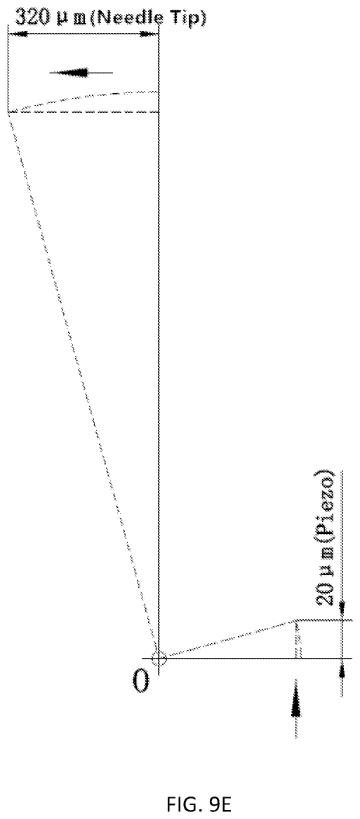

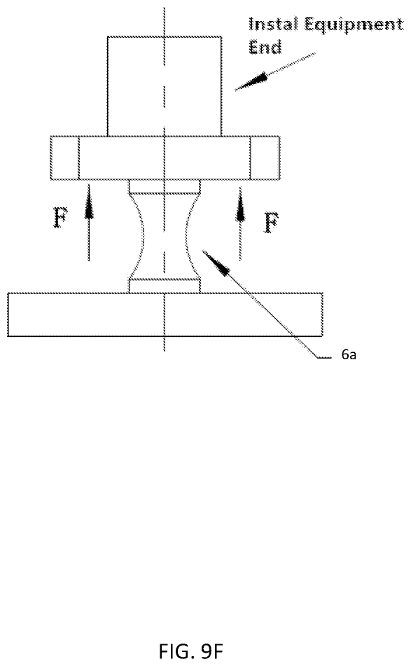

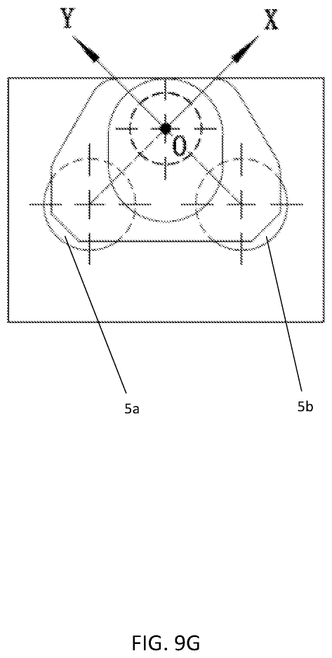

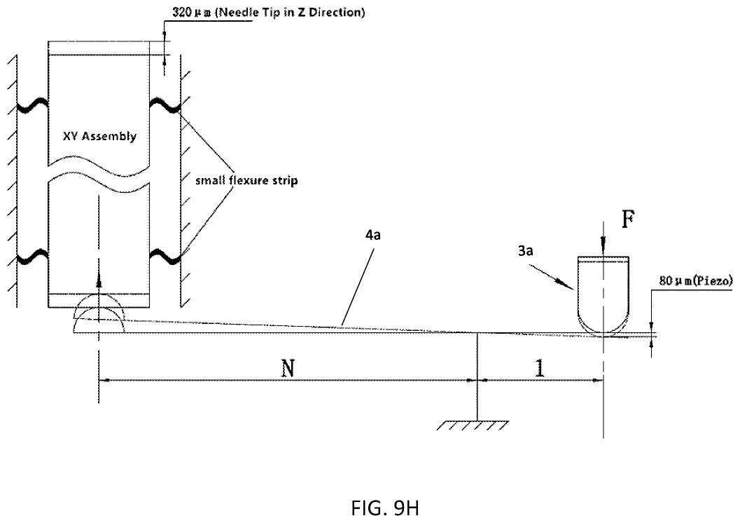

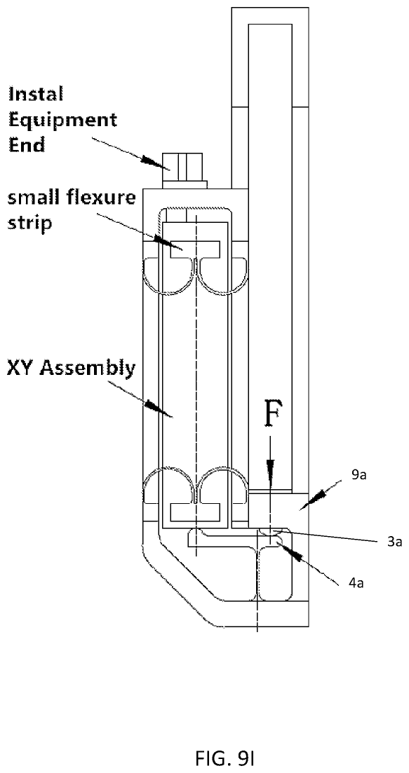

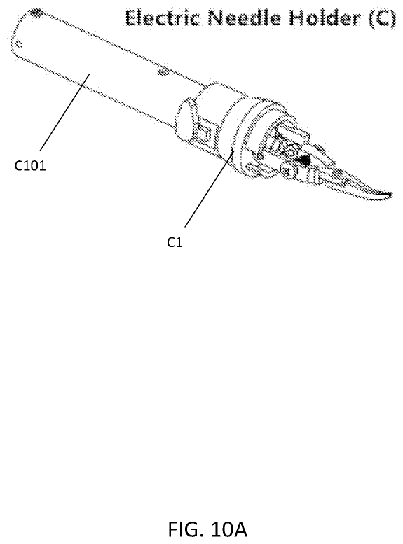

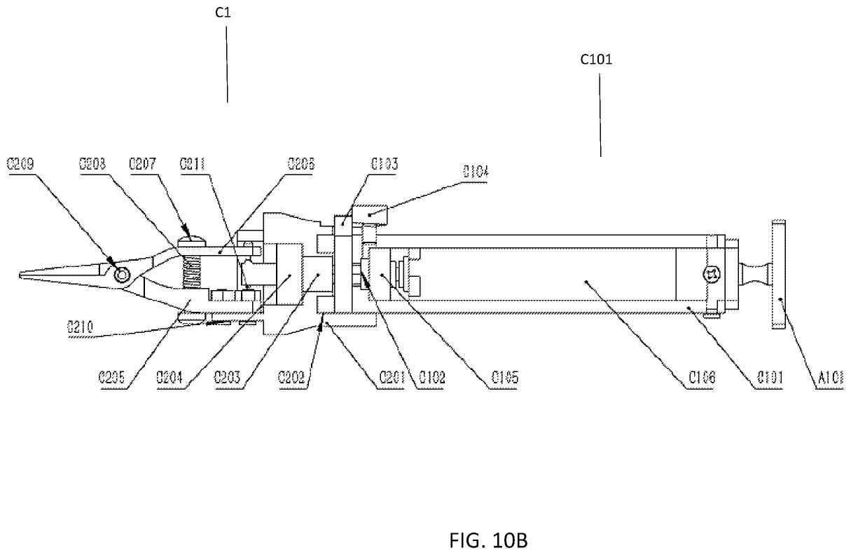

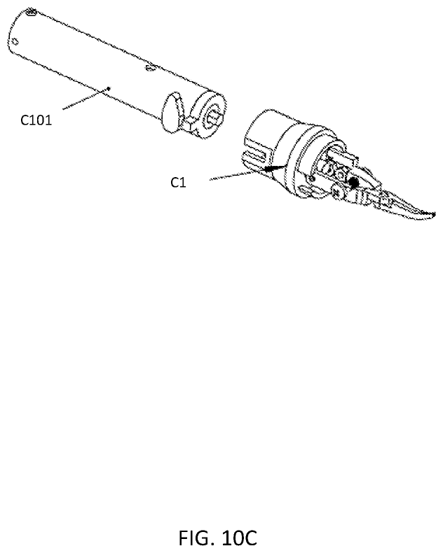

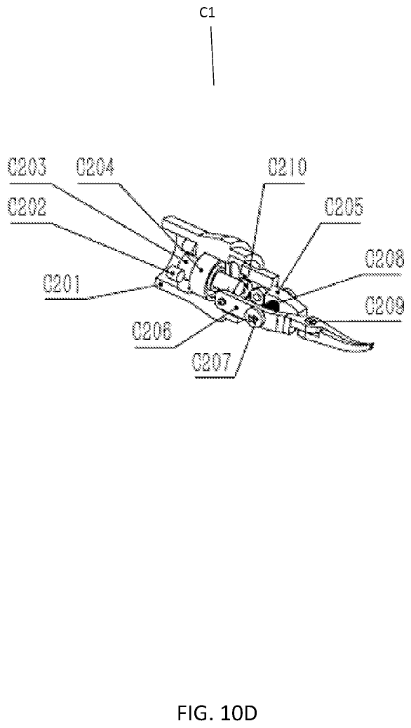

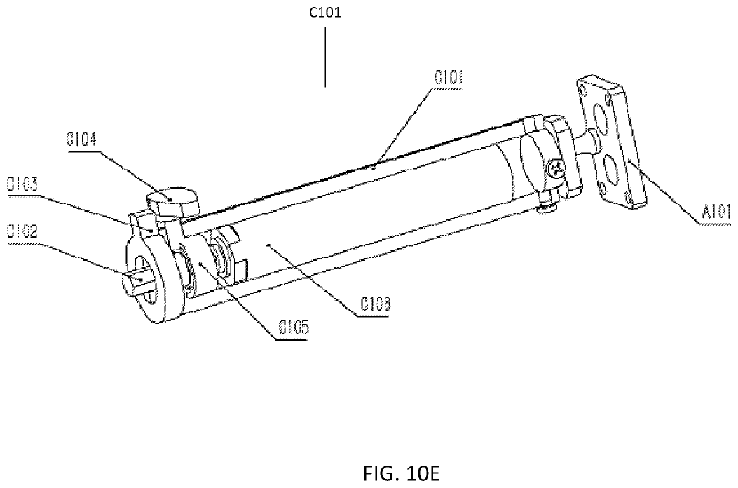

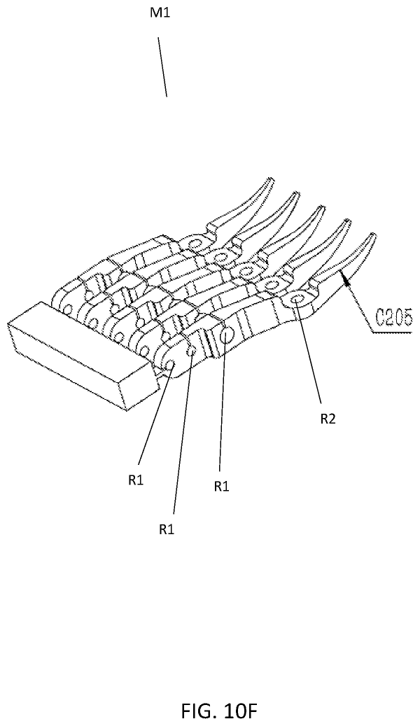

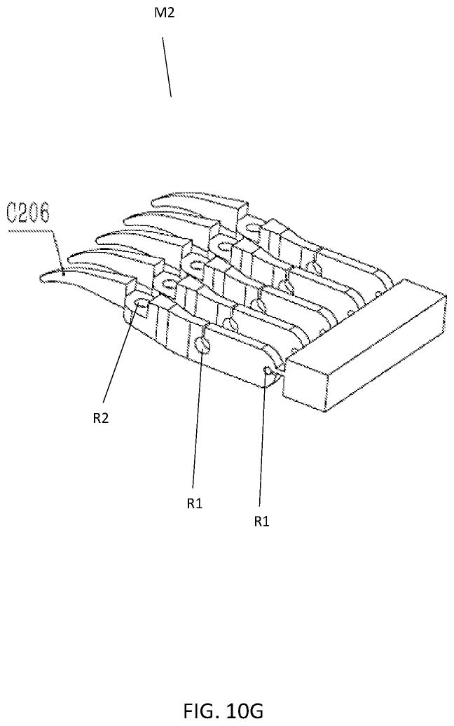

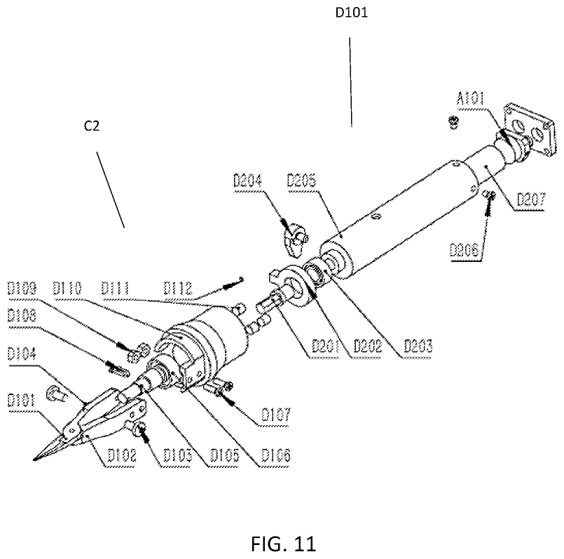

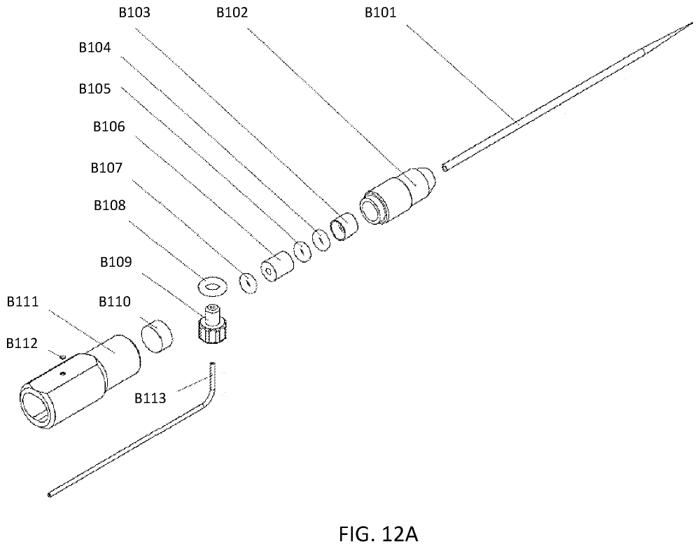

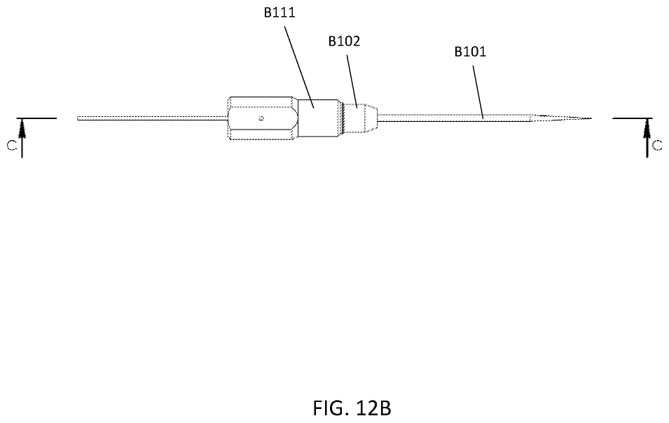

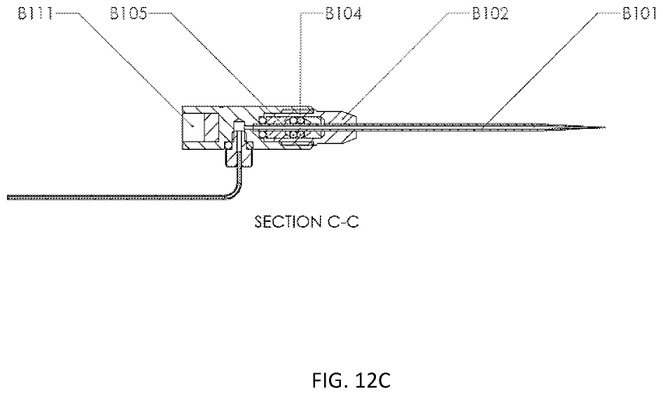

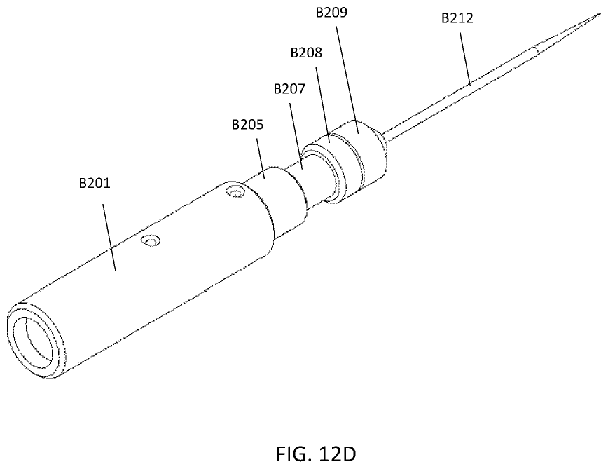

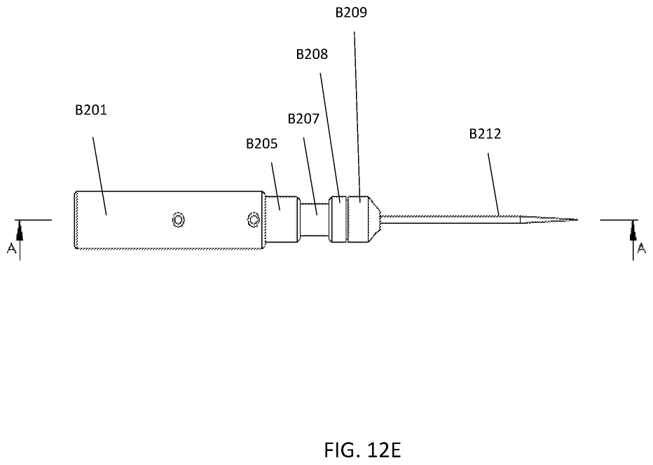

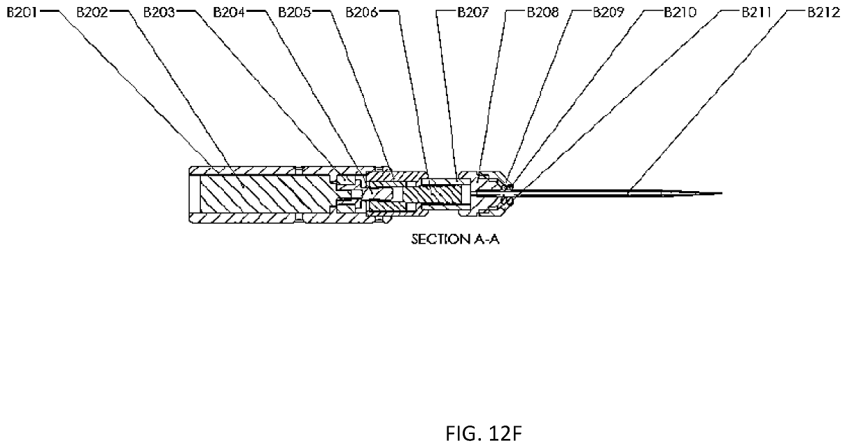

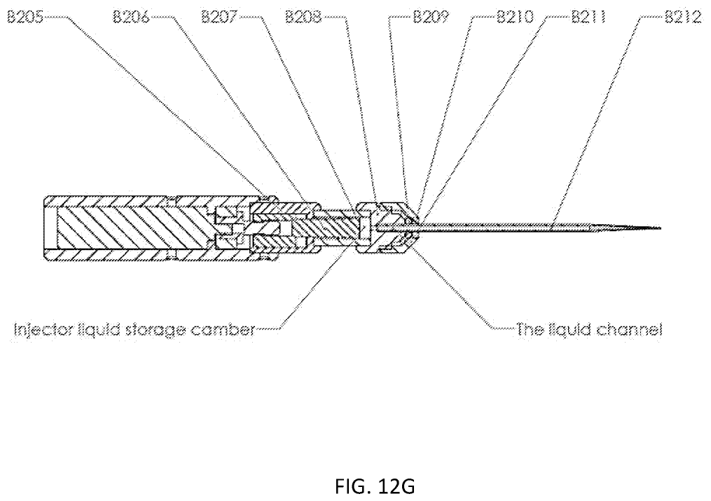

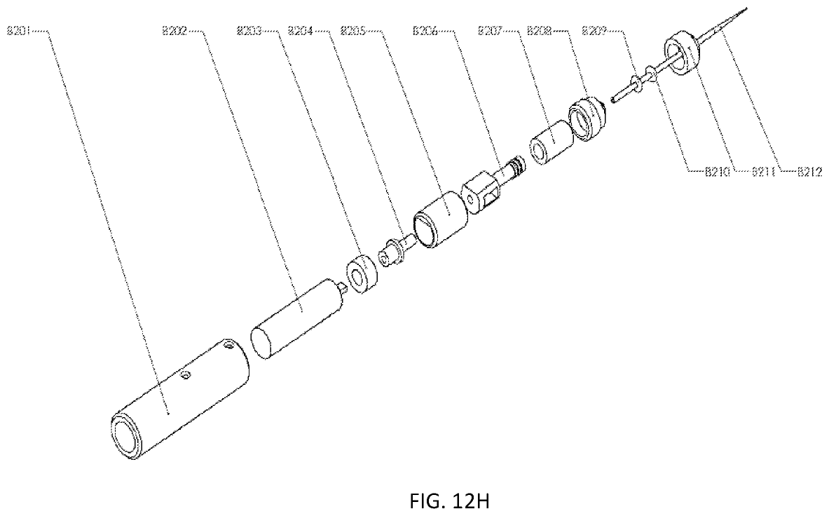

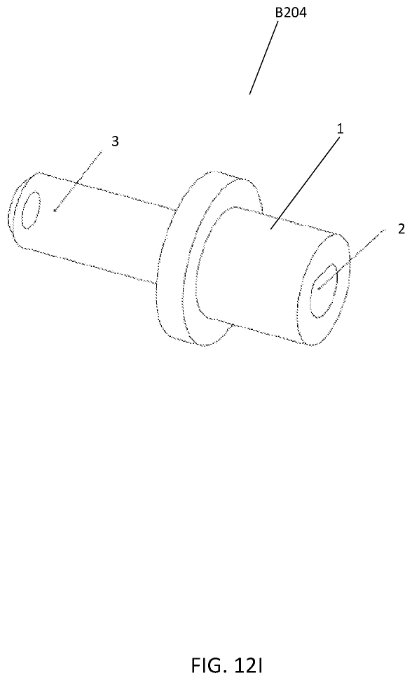

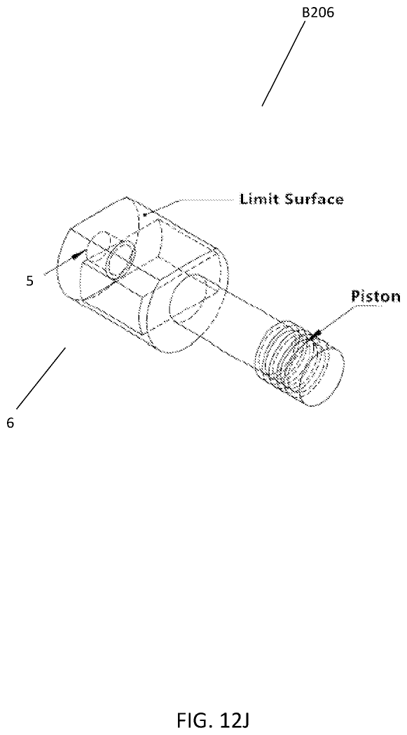

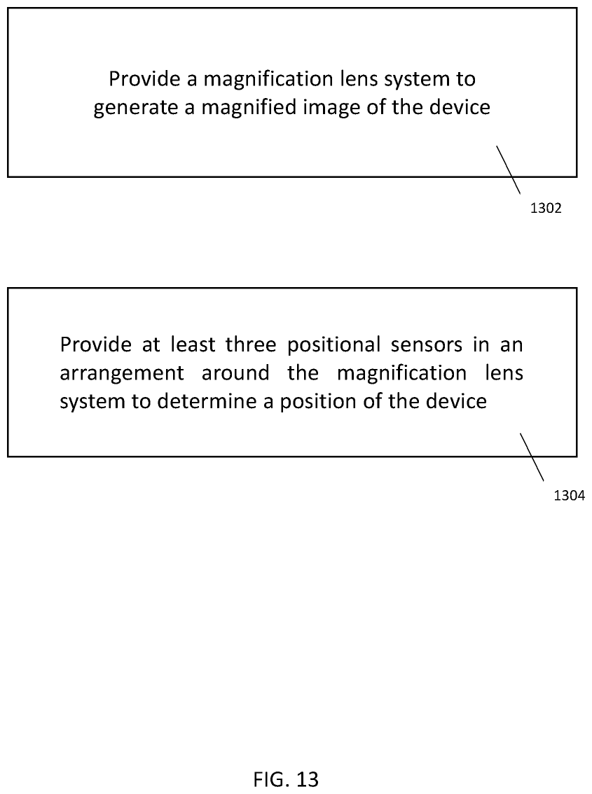

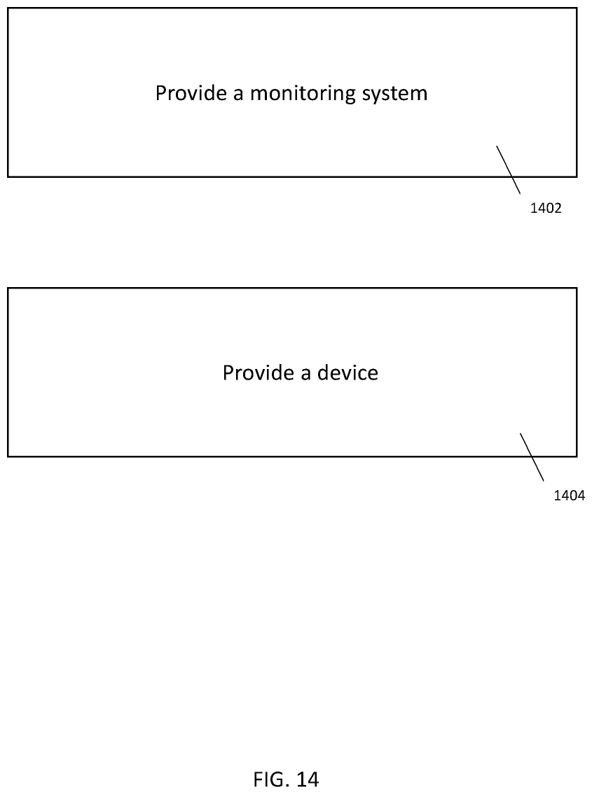

FIGS. 7A-7D show various examples of a plurality of positional sensors (e.g. two positional sensors or at least three positional sensors) configured to monitor and detect a device include at least three light sources, according to various embodiments.

FIGS. 7A-7B show a set-up including two positional sensors (PSD1 and PSD2), wherein the two positional sensors PSD1 and PSD2 are immovable relative to a support to which the positional sensors PSD1 and PSD2 are attached, and the limitations thereof. FIG. 7A illustrates that when the device 70 is in a first position and/or first orientation, each of the at least two positional sensors may detect (or receive) a light emitted from each of the at least three light sources of the device 70 according to various embodiments. FIG. 7B illustrates that when the device 70 is in a second position and/or orientation, only one positional sensor PSD1 may detect (or receive) a light emitted from all three light sources of the device 70 according to various embodiments. The other positional sensor PSD2 may not capture at least one light source of the device 70 which may be occluded by a body of the device 70. Thus, in FIG. 7B, a three-dimensional position and/or a three-dimensional orientation of the device cannot be detected or monitored (or determined) by the set-up including at most two immovable positional sensors (PSD1 and PSD2), since the three-dimensional position and/or the three-dimensional orientation of the device cannot be determined by a monocular vision system (i.e. only one positional sensor PSD1), but requires at least a binocular vision system (e.g. at least two positional sensors to detect or receive a light emitted from all three light sources of the device).

FIGS. 7C-7D show at least three positional sensors of a monitoring system comprising including at least three positional sensors, according to various embodiments. FIG. 7C illustrates that when the device 70 is in a first position and/or first orientation (which may or may not be similar to the first position and/or first orientation in the examples in FIGS. 7A-7B), each of the at least three positional sensors (PSD1, PSD2, PSD3) may detect (or receive) a light emitted from each of the at least three light sources of the device 70, according to various embodiments. FIG. 7D illustrates that when the device 70 is in a second position and/or orientation (which may or may not be similar to the first position and/or first orientation in the examples in FIGS. 7A-7B), where the body of the device 70 may occlude (or block) a light of at least one light source from being detected by at least one positional sensor (PSD1) (e.g. a light of at least one light source is not within a line of sight of at least one positional sensor), at least two other positional sensors (PSD2, PSD3) may detect (or receive) a light emitted from all three light sources of the device 70 and thereafter determine a three-dimensional position and/or a three-dimensional orientation of the device according to various embodiments.

Accordingly, according to various embodiments, when at least three positional sensors of a monitoring system are focusing on a space (e.g. when a working space of each of the at least three positional sensors overlap), a workspace of the monitoring system may have a similar size as a workspace of a set-up including at most two positional sensors, but the monitoring system including at least three positional sensors may be able to monitor and detect a higher number of different or varying poses (e.g. position and/or orientation) of the device as well as more flexible poses (e.g. a larger change in position and/or orientation) of the device than a set-up including at most two positional sensors, since a workspace of a monitoring system including at least three positional sensors includes all permutations of every pair or every possible grouping of at least two position sensors of the at least three positional sensors of the monitoring system.

Accordingly, according to various embodiments, when dealing with certain positions and/or orientations of the device, where only one positional sensor (“working positional sensor”) of at least three positional sensors of a monitoring system may immediately detect signals from all of at least three LEDs of a device, at the first instance when the device is placed within a workspace (e.g. location for placing a device for monitoring of the device) of the monitoring system, another positional sensor of the at least three positional sensors may be configured to adjust (by manual or automatic means) a position (e.g. front, back, up, down, left or right position) and/or an orientation (e.g. an elevation angle, an azimuth angle and/or a torsion angle) relative to a respective sensor support and/or a position relative to a circular support of the monitoring system, to form a pair (e.g. 2-PSD system) with the working positional sensor, such that the other positional sensor may detect signals from all of the at least three LEDs of the device, to ensure that signals from all of the at least three LEDs may be detected by at least two positional sensors of at least three positional sensors of the monitoring system in order to determine a three-dimensional position and/or a three-dimensional orientation of the device.

Thus, according to various embodiments, a line of sight of each positional sensor of a monitoring system may be changeable (by manual or automatic means) at any point of time to ensure that signals (e.g. light) from all of at least three light sources of a device may be detected by at least two positional sensors of at least three positional sensors of a monitoring system to enable a determination of a three-dimensional position and/or a three-dimensional orientation of the device. In other words, according to various embodiments, a sightline occlusion of a positional sensor (e.g. of at least three positional sensor) may be avoided by movement or adjustment (by any suitable means) of a position and/or an orientation of the positional sensor.

According to various embodiments, a monitoring system may include a plurality of positional sensors (e.g. four, five, six, seven, eight or more than eight positional sensors).

Accordingly, according to various embodiments, each pair of positional sensors (or each group of two positional sensors) of the plurality of positional sensors (e.g. four, five, six, seven, eight or more than eight positional sensors) may focus on a respective space that is different from a space that is focused on by another pair or pairs (or all of the other pairs) of the plurality of positional sensors (e.g. four, five, six, seven, eight or more than eight positional sensors). Accordingly, the monitoring system may have multiple views (e.g. 2, 3, 4 or more than 4 views) for monitoring (or detecting) a position and/or an orientation (e.g. three-dimensional position and/or three-dimensional orientation) of a device and/or a further device. By way of example, when the number of positional sensors is an odd number (e.g. seven positional sensors), after all the possible pairs of positional sensors have been formed, the remaining positional sensor may focus on a space that is focused on by any of the formed pairs of positional sensors.

It may also envisioned that in various embodiments, each (e.g. one) positional sensor of the plurality of positional sensors (e.g. four, five, six, seven, eight or more than eight positional sensors) may focus on a respective space that is different from a space that is focused on by another (e.g. one) positional sensor of the plurality of positional sensors (e.g. four, five, six, seven, eight or more than eight positional sensors). Accordingly, the monitoring system may have multiple views (e.g. 2, 3, 4 or more than 4 views) for monitoring (or detecting) a position of a device and/or a further device.

According to various embodiments, the plurality of positional sensors (e.g. at least three positional sensors) of the monitoring system may move together (in tandem) with the magnification lens system (e.g. microscope) of the monitoring system whenever the magnification lens system is moved, and the relative positions of each positional sensor of the plurality of positional sensors (e.g. at least three positional sensors) of the monitoring system may be adjustable (e.g. by manual or motorized means).

According to various embodiments, since visible light signal may be interfered by ambient light and may not be easily detectable, since there may be high signal-to-noise ratio, the plurality of light sources of the device may be configured to emit infrared light. Further, the plurality of positional sensors (e.g. at least three positional sensors) of the monitoring system may be configured to detect an infrared light (e.g. the infrared light of the plurality of light sources of the device).

Accordingly, according to various embodiments, at least three light sources (e.g. light-emitting diodes (LEDs)), which may emit a predetermined wavelength or range of wavelengths of light (e.g. infrared) having a controlled (or predetermined) frequency (e.g. that may be higher than a sampling frequency), may be mounted on a surface (e.g. handle) of the device (e.g. a handheld surgical tool). Accordingly, according to various embodiments, each of the at least three LEDs on the device may emit signals (e.g. infrared signals) at a frequency of at least 400 Hz or more.

According to various embodiments, the at least three LEDs may be mounted on a surface of a rigid body of the device. According to various embodiments, the device may have a tubular body with an outer surface. Accordingly, each of the at least three LEDs may be mounted on any portion on the outer surface at any position around the tubular body.

For example, FIG. 8A shows a device 80 and a further device 81, according to various embodiments.

Referring to FIG. 8A, a first LED (LED1) and a second LED (LED2) may be mounted on a surface of a middle portion of a handle of the device 80 and a third LED (LED3) may be mounted on a surface of a bottom portion of the handle of the device 80. The middle portion may be located at the center of the device 80, and the bottom portion may be closer to a working tip 85 of the device 80 than the middle portion. As shown in FIG. 8A, the first LED (LED1), the second LED (LED2) and the third LED (LED3) may form the edges of a geometric triangular shape.

According to various embodiments, the at least three LEDs (e.g. LED1, LED2, LED3) may provide three points of spatial information (or spatial information of three points) of the device 80 and may provide information of a position and/or an orientation of the device 80, to a monitoring system.

According to various embodiments, a plurality of devices may be provided. According to various embodiments, each device of the plurality of devices may include a plurality of LEDs (e.g. infrared LEDs) on a surface of each device. For example, as shown in FIG. 8A, a further device 81 may be provided. As shown in FIG. 8A, the device 80 may be a left handheld device and the further device 81 may be a right handheld device. The further device 81 may, likewise to device 80, include a plurality of plurality of LEDs (LED4, LED5, LED6). As shown, LED4 and LED5 may be mounted on the surface of a middle portion of a handle of the further device 81 and LED6 may be mounted on the surface of a bottom portion of the handle of the device.

According to various embodiments, in order to distinguish between the different LEDs of each device of the plurality of devices and/or between the different LEDs of different devices, each LED of the plurality of devices may be configured to flash successively within at least one period of time, during operation or use of the monitoring system for monitoring the device. For example, FIG. 8B shows a time flow of action and inaction of a plurality of LEDs for calibration according to various embodiments. With reference to FIG. 8B, according to various embodiments, within a first period of time (e.g. 1/400 s), the time flow of action and inaction of the LEDs may be: LED1 lights on, LED1 lights off, LED2 lights on, LED2 lights off, LED3 lights on, LED3 lights off, LED4 lights on, LED4 lights off, LED5 lights on, LED5 lights off, LED6 lights on, LED6 lights off. In other words, within the first period of time, the LEDs may operate in this succession (e.g. sequence or order): LED1 may turn on flash for a first segment (or period) of time (e.g. <= 1/2400 s); LED1 may turn off; LED2 may turn on flash for a second segment of time (e.g. <= 1/2400 s); LED2 may turn off; LED3 may turn on flash for a third segment of time (e.g. <= 1/2400 s); LED3 may turn off; LED4 may turn on flash for a fourth segment of time (e.g. <= 1/2400 s); LED4 may turn off; LED5 may turn on flash for a fifth segment of time (e.g. <= 1/2400 s); LED5 may turn off; LED6 may turn on flash for a sixth segment of time (e.g. <= 1/2400 s); LED6 may turn off. According to various embodiments, at any given time, there may only be one LED turned on. In other words, when one LED of a plurality of LEDs is turned on, each of the other LEDs of the plurality of LEDs would be turned off. According to various embodiments, within a period of time of 1/400 s, each segment of time during which each LED is turned on is no more than 1/2400 s, to ensure that light signals from each successive LEDs (e.g. of a plurality of six LEDs) do not interfere with one other.

According to various embodiments, there may be more than one period of time (e.g. a second period, a third period, a further period) (of e.g. 1/400 s), and in each period of time (e.g. 1/400 s), the LEDs may operate in the same succession (e.g. sequence or order) as the succession in the first period of time.

Further, according to various embodiments, to ensure that the signals from the plurality of light sources (e.g. three LEDs) of each device (or, for example, the plurality of light sources LEDs 1-6 of the device and the further device in FIG. 8A) are being detected by at least at least two positional sensors in each period of time, the sampling rate of each positional sensor may be at least 6 times more than (or at least 6 folds of, or having a magnitude of at least 6 time greater than) (e.g. 2400 Hz) a frequency (e.g. 1/2400 s) of each LED.

According to various embodiments, in order to achieve a detection of precise three-dimensional information (e.g. of a device), a system calibration may be implemented to establish a spatial relationship between each positional sensor of a plurality of positional sensors of a monitoring system.

According to various embodiments, a calibration theory similar to a calibration theory of a binocular vision system may be adopted for the system calibration.

According to various embodiments, conventional calibration methods are used for typical cameras, which may be an imaging device. In contrast, a positional sensor, for example, a PSD, may not be configured to generate normal images. Instead, the positional sensor (e.g. PSD) may be configured to provide position information of an incident light spot.

According to various embodiments, Zhang's theory may be utilized to finish calibration. Zhang's theory may be understood that be a calibration method: “A Flexible New Technique for Camera Calibration”, proposed by Zhang. Z in 1998. According to the calibration method, a checkerboard (e.g. of squares) is placed at several different positions within a field of view of a camera. Physical dimensions of the squares on the checkerboard may be provided. The corners of the squares on the checkerboard are captured by the camera at each position of the several different positions. Homography matrices between the image plane of the camera and the plane of the checkerboard (in the several different positions) are established. Subsequently, the camera's intrinsic and extrinsic parameters may be calculated via spatial geometry, singular-value decomposition of matrix and non-linear optimization etc. Accordingly, according to various embodiments, a calibration board (or device) including an LED array may be provided. For example, the calibration board may include a 7*7 rectangular LED array (e.g. 49 LEDs), in which each LED emits a light (e.g. infrared light) of a same wavelength as that of a LED (or a plurality of LEDs) on a manipulator.

Accordingly, according to various embodiments, a calibration sequence may be provided. During the calibration sequence the calibration board may be first fixed at an initial position. Subsequently, each LED on this array turn on and turn off, consecutively and/or sequentially (e.g. one after another and/or one at each time), according to a specific sequence. Accordingly, in any instant moment, only one LED may emit a light (or signal). In other words, when one LED of a plurality of LEDs is turned on, each of the other LEDs of the plurality of LEDs would be turned off. According to various embodiments, each positional sensor may be configured to detect (or receive) the signals of from each of the 49 LEDs of the calibration board. After every signal from each of the 49 LEDs of the calibration board has been recorded by a positional sensor of a plurality of positional sensors, the calibration board may be moved to a different (new) position from the initial position.

When the calibration board in the different (new) position, the calibration sequence above may be repeated for the positional sensor (i.e. the positional sensor in question).

Finally, after a predetermined number of the calibration sequences has been performed for a positional sensor (in other words, after the positional sensor has acquired sufficient data), Zhang's theory (or algorithm) may be utilized to calculate intrinsic and extrinsic parameters as well as distortion coefficients of the positional sensor.

Embodiments of a device, according to various embodiments, would now be elaborated.

According to various embodiments, the device may be a handheld device that may be configured to cancel or provide compensation for physiological tremor of a user's hand, to ensure that any erroneous displacement at an end effector (e.g. tooltip) of the device falls within (or remains within) an acceptable range, for example, when a surgeon performs microsurgery operations using a microscope.

According to various embodiments, the device may be configured to detect and collect a data, for example, a range and or a frequency of a human hand tremor, and the device may be configured to thereafter transmit the data to a computer. The computer may, in turn, be configured to predict an erroneous displacement (e.g. a potential erroneous displacement) at an end effector of a device through machine learning, based on the data that the computer receives from the device. Accordingly, the device may be configured to compensate, in advance, an erroneous displacement (e.g. tremor) at the end effector of the device through a control algorithm, such that the erroneous displacement at the end effector of the device is controlled and kept within an acceptable range.

FIG. 9A shows an exploded view of a device according to various embodiments. FIG. 9B is an assembled view of the device shown in FIG. 9A according to various embodiments. FIG. 9C is an exterior view of the device shown in FIG. 9A according to various embodiments. FIG. 9D shows a see-through view of the device according to various embodiments. FIG. 9E illustrates the principle of the degrees of freedom along the x-axis and the y-axis of an end effector of the device according to various embodiments. FIG. 9F illustrates the principle of the degrees of freedom along the x-axis and the y-axis of an end effector of the device, based on a x-y-axis frame, according to various embodiments. FIG. 9G illustrates the principle of the degrees of freedom along the x-axis and the y-axis of an end effector of the device, based on the x-axis pin and the y-axis pin, according to various embodiments. FIG. 9H illustrates the principle of the degrees of freedom along the z-axis of an end effector of the device according to various embodiments. FIG. 9I shows a schematic side view of the device according to various embodiments.

According to various embodiments, with reference to FIG. 9A, the device may include a power circuit board 1a, an end effector 2a, a z-axis pin 3a, a z-axis lever 4a, a x-axis pin 5a, a y-axis pin 5b, a x-y-axis lever 6a, a x-y-axis frame 7a including at least two flexure structures on each side of the x-y-axis frame 7a, a x-y-axis piezo actuator 8a, a casing 9a, a z-axis piezo actuator 10a, and a mainframe 11a.

According to various embodiments, the power circuit board 1a, the end effector 2a, the z-axis pin 3a, the z-axis lever 4a, the x-axis pin 5a, the y-axis pin 5b, the x-y-axis lever 6a, the x-y-axis frame 7a, the x-y-axis piezo actuator 8a, the casing 9a and the z-axis piezo actuator 10a may respectively be coupled to or placed within the mainframe 11a. According to various embodiments, the z-axis pin 3a may be coupled to the z-axis lever 4a. According to various embodiments, the x-axis pin 5a and the y-axis pin 5b may respectively be coupled to the x-y-axis lever 6a.

In this specification, reference to an XY assembly may be reference to a combination of the following components: the x-axis pin 5a, the y-axis pin 5b, the x-y-axis lever 6a, the x-y-axis frame 7a including at least two flexure structures on each side of the x-y-axis frame 7a and the x-y-axis piezo actuator 8a.

In order to minimize the number of installation steps involved in joining the parts of the device and minimize error which may occur during an installation of the device or such a device, the XY assembly may be provided as an integral structure, for example, a single structure, such that no installation or coupling of the parts is required by an end-user of the device. The integral structure of the XY assembly may be manufactured by any suitable technique (e.g. Laser bonding, interference fit etc.).

With reference to FIG. 9B, the device without an end effector attached may have the following boundary dimensions: a width of approximately 14 millimetres (mm); a height of approximately 26-27 mm; a length of approximately 76-77 mm. Further, the device without an end effector attached may have a weight of approximately 50 grams or less. It may be envisioned that in various embodiments, the device, with or without an end effector attached, may have any suitable width, any suitable height, any suitable length, and any suitable weight.

According to various embodiments, the end effector of the device may have three degrees of freedom in space. For example, with reference to FIG. 9C, the end effector of the device may move along a x-axis, a y-axis and a z-axis.

Accordingly, since a physiological tremor of a person's hand may cause a displacement (e.g. of a device) of within approximately 300 micrometers (μm) (or may move within an approximate distance of 300 μm), the device may drive the end effector, having three degrees of freedom in space, with a 300 μm stroke along each axis of the each of the three degrees of freedom in space (e.g. x, y and z degrees of freedom), which may counter (or attenuate) the displacement caused by the physiological tremor of the hand.

The principle of the degrees of freedom along the x-axis and the y-axis of the end effector of the device will now be elaborated with reference to FIGS. 9E-9I.

According to various embodiments, the x-y-axis piezo actuator 8a may be configured to move the end effector along the x-axis and/or the y-axis of the end effector. The x-y-axis piezo actuator 8a may have a 25 μm stroke (e.g. along each direction in the x-axis and/or the y-axis). To achieve a 300 μm stroke at the end effector (e.g. at the tip of the end effector), a displacement of the end effector caused by the x-y-axis piezo actuator 8a may be magnified according to various embodiments. For example, a magnification of the displacement of the end effector, caused by x-y-axis piezo actuator 8a, may be proportional to (or based on) a length of the end effector and/or proportional to a distance between a tip of the x-axis pin 5a and/or the y-axis pin 5b and a center line of the x-y-axis lever 6a (e.g. distance from the x-y-axis lever 6a to the tip of the x-axis pin 5a and/or the tip of the y-axis pin 5b).

According to various embodiments, the core part is the x-y-axis lever 6a and the x-y-lever 6a may be made of Titanium Alloy material.

Further, according to various embodiments, the flexure strips and z-axis lever 4a may be made from a material such as Titanium Alloy or any other suitable material with a property of high elasticity and a property of high yield strength.

The principle of the degrees of freedom along the z-axis of the end effector of the device will now be elaborated with reference to FIGS. 9E-9I.

According to various embodiments, the z-axis piezo actuator 10a may be configured to move the end effector along the z-axis of the end effector. The z-axis piezo actuator 10a may have a 90 μm stroke (e.g. along a z-axis direction). According to various embodiments, to achieve a 300 μm stroke at the end effector, a displacement of the end effector caused by z-axis piezo actuator 10a may be magnified. According to various embodiments, a magnification of the displacement of the end effector, caused by x-y-axis piezo actuator 8a, may be based on a lever principle and based on the XY assembly.

For example, referring to FIG. 9H, the z-axis pin 3a may be coupled to the z-axis lever 4a which is, in turn, coupled to a base of the XY assembly which is, in turn, coupled to end effector 2a. Movement of the z-axis pin 3a (e.g. by the z-axis piezo actuator 10a) may cause a swing motion of the z-axis lever 4a which, in turn, moves the end effector 2a via the XY assembly. Accordingly, a displacement caused by the z-axis piezo actuator 10a may be magnified by way of the mechanical advantage that may be provided by the z-axis lever 4a. As the z-axis lever 4a performs the swing motion to move the end effector 2a along the z-axis direction, the group of four flexure strips of the x-y-axis frame 7a (i.e. at least two flexure structures on each side of the x-y-axis frame 7a), of the XY assembly, may attenuate or prevent any displacement of the end effector 2a along the x-axis direction and/or along the y-axis direction. Accordingly, the group of four flexure strips of the x-y-axis frame 7a may restrict (or confine) the XY assembly to only move along the z-axis when the z-axis lever 4a causes the XY assembly to move.

According to various embodiments, the device may be a surgical tool (e.g. a handheld surgical tool). According to various embodiments, the surgical tool may be a holder, a single-blade cutter (e.g. knife), a dual-blade cutter (e.g. scissors), a fluid injector (e.g. syringe) or any other suitable surgical tool.

According to various embodiments, the device may be a motorized surgical tool. The motorized surgical tool may include a motor assembly part and a tool assembly part.

According to various embodiments, the motor assembly part may include a motor base, a motor, a motor output shaft, a rotary knob, a ferromagnetic component and a bearing.

According to various embodiments, the tool assembly part may be a clamping assembly part (e.g. an electric needle holder with curved forceps, an electric needle holder with straight pliers, a scissors assembly part (e.g. an electric microscopic scissors, an electric tweezers), an injection assembly part (e.g. an electric injection syringe) or any other suitable electric tool.

According to various embodiments, the motor assembly part may be attached to the tool assembly part or to any other tools or instruments (e.g. needle holder with curved forceps, needle holder with straight pliers, microscopic scissors, tweezers, injection syringe etc.), such that the motor assembly part may manipulate the tool assembly part or any other tool or instrument.

Accordingly to various embodiments, the device may be any of a motorized needle holder with curved forceps, a motorized needle holder with straight pliers, a motorized microscopic scissors, a motorized tweezers, a motorized injection syringe etc.

As shown in FIGS. 10A-10G, the device is illustrated as a motorized needle holder. FIG. 10A shows a perspective view of the motorized needle holder according to various embodiments. FIG. 10B shows a side view of the motorized needle holder according to various embodiments. FIG. 10C shows a view of the motorized needle holder of FIG. 10A according to various embodiments in which the clamping assembly part C1 is separated from the motor assembly part. FIG. 10D shows the clamping assembly part C1 of the motorized needle holder according to various embodiments. FIG. 10E shows the motor assembly part of the motorized needle holder according to various embodiments. FIG. 10F shows a plurality of first forcep slices of a motorized needle holder according to various embodiments. FIG. 10G shows a plurality of first forcep slices of a motorized needle holder according to various embodiments.

The motorized needle holder may include a xy deformation structure A101, a motor assembly part A102 attached to the xy deformation structure A101, and a clamping assembly part C1 coupled to the motor assembly part C101.

As shown in FIGS. 10B and 10E, according to various embodiments, the motor assembly part C101 may include a motor base C101, a motor C106, a motor output shaft C102, a ferromagnetic component C103 (e.g. 440C Stainless Iron), a rotary knob C104 and a bearing C105.

As shown in FIGS. 10B and 10D, according to various embodiments, the clamping assembly part C1 may include a jacket shell C201, a magnet C202, a reel C203, a bearing C204, a first forcep slice C205, a second forcep slice C206, a spring plate C207, a return spring C208, an assembly pin C209, a lock screw module C210 and a bracing wire C211.

According to various embodiments, the motor assembly part C101 may be configured to produce a rotary motion by way of the motor C106 and may be further configured to transmit the rotary motion of the motor C106 by way of a motor output shaft C102 to the reel C203 of the clamping assembly part C1. In other words, the motor C106 of the motor assembly part C101 may be configured to rotate the reel C203 of the clamping assembly part C1 via the motor output shaft C102. The reel C203 may be connected to the second forcep slice C206 via the bracing wire C11. According to various embodiments, one forcep slice (e.g. the first forcep slice C205) is immovable relative to the motorized needle holder. Accordingly, the other forcep slice (e.g. the second forcep slice C206) may be movable relative to the motorized needle holder.

By having only one movable forcep slice and having another forcep slice that is immovable, a closing or an opening action of the two forcep slices, which are effected by the motor and the reel, would not produce any shearing force or torsion between the two forcep slices during the closing or the opening action.

According to various embodiments, when the reel is rotated in a first direction, the movable forcep slice (e.g. the second forcep slice C206) may be pulled towards the immovable forcep slice (e.g. the first forcep slice C205), and accordingly, a clamping function of the motorized needle holder is achieved.

According to various embodiments, an end of the motor output shaft C102 includes a tetrahedral shape (or other polyhedral shapes), which may be inserted into a corresponding receiving portion (e.g. recess) of the reel C203. Accordingly, when the end of the motor output shaft C102 is inserted into the receiving portion of the reel C203, the motor C106 may rotate the drive motor output shaft C102 which, in turn, causes the reel C203 to rotate.

In other words, a bracing wire c211 is wound around the reel C203 and the bracing wire c211 is further coupled to a tail end of the second forcep slice. According to various embodiments, the reel C203 is positioned between a tail end of the first forcep slice C205 and a tail end of the second forcep slice C206. Accordingly, when the reel C203 is rotated in a first direction, the reel C203 winds the bracing wire c211 which, in turn, pulls the second forcep slice C206 towards a central longitudinal axis of the motorized needle holder (i.e. towards the reel C203 and toward the first forcep slice C205), thereby causing the first and the second forceps slices to close. Accordingly, when reel C203 rotated in a second direction (e.g. opposite direction), the reel C203 unwinds the bracing wire c211 and, at the same time, the return spring C208 may push the second forcep slice C206 away from the central longitudinal axis of the motorized needle holder (i.e. away from the reel C203 and away from the first forcep slice C205), thereby causing the first and the second forceps slices to open.

In other words, according to various embodiments, a motorized device may be provided. The motorized device may include a motor assembly part including a motor coupled to a motor output shaft having a protrusion. The motorized device may further include a tool assembly part including a scissors mechanism, a reel and a return spring. The scissors mechanism may be two longitudinal elements pivotally coupled at a middle portion of each of the two longitudinal elements. The tool assembly part may further include a bracing wire that may wound around the reel and the bracing wire is may be further coupled to a tail end of one longitudinal element (e.g. a first longitudinal element) of the scissors mechanism. According to various embodiments, the reel is positioned between a tail end of each of the two longitudinal elements of the scissors mechanism. According to various embodiments, the one longitudinal element (e.g. the first longitudinal element) may be movable relative to the tool assembly part, and the other longitudinal element (e.g. the second longitudinal element) may be immovable relative to the tool assembly part. The reel may include a recess configured to receive the protrusion of the motor output shaft of the motor assembly part. Accordingly, when the protrusion of the motor output shaft of the motor assembly part is inserted into the recess of the reel, the motor may rotate the reel via the motor output shaft. When the reel is rotated in a first direction, the reel winds the bracing wire which, in turn, pulls the one longitudinal element (e.g. the first longitudinal element) towards a central longitudinal axis of the motorized device (i.e. towards the reel and toward the other longitudinal element), thereby causing the two longitudinal elements of the scissors mechanism to close. Accordingly, when reel rotated in a second direction (e.g. opposite direction), the reel unwinds the bracing wire and, at the same time, the return spring may push the one longitudinal element away from the central longitudinal axis of the motorized device (i.e. away from the reel and away from the other longitudinal element), thereby causing the two longitudinal elements of the scissors mechanism to open.

According to various embodiments, the clamping assembly part C1 may be configured to be easily and quickly inserted into (or coupled to) the motor assembly part C101. For example, the magnet C202 of the clamping assembly part C1 may be attracted to the ferromagnetic component C103, thereby providing a magnetic coupling that joins the clamping assembly part C1 and the motor assembly part C101 together. The magnetic coupling (or the connection) between the clamping assembly part C1 and the motor assembly part C101 may be further secured by way of a rotary knob which may be configured to lock the clamping assembly part C1 and the motor assembly part C101 together.

According to various embodiments, the clamping assembly part C1 may be configured to be disposable.

According to various embodiments, the motor assembly part C101 may be configured to be attached (e.g. fixed) to a main instrument handle and may further be configured to be robust (e.g. for repeated use or for use indefinitely).

According to various embodiments, a method of manufacturing a plurality of tool assemblies (e.g. needle holder assemblies) is provided, for example, by a single wire-cutting technique on a material and drilling of at least two holes on the material in a crosswise manner. As shown in the FIGS. 10F and 10G, at least one first recess (e.g. hole) R1 may be created (e.g. drilled) along a longitudinal axis of a first material M1 and a second material M2, and at least one second recess R2 may be created along a lateral axis (i.e. perpendicular to the longitudinal axis) of the first material M1 and the second material M2, to manufacture a plurality of first forcep slices C205 from material M1 and a plurality of second forcep slices C206 from material M2. Accordingly, a needle holder structure of the motorized needle holder may be designed to be manufactured from at least one material by a single wire-electrode cutting technique. Accordingly, a plurality of sets of the needle holder structure may be manufactured at any one time, thereby reducing fabrication costs (i.e. cost-efficient) and decreasing fabrication difficulty (i.e. easy to manufacture).

FIG. 11 shows a view of a disassembled motorized microsurgery scissors according to various embodiments. As shown in FIG. 11, the device is illustrated as a motorized microsurgery scissors.

The motorized microsurgery scissors may include a xy deformation structure A101, a motor assembly part C101 which is attached to the xy deformation structure A101, and a scissors assembly part C2 coupled to the motor assembly part D101.

As shown in FIG. 11, according to various embodiments, the motor assembly part D101 may include a motor output shaft D201, a ferromagnetic component D202 (e.g. 440C Stainless Iron), a bearing D203, a rotary knob D204, a motor base D205, a set screw D206 and a motor D207 (e.g. micro motor).

As shown in FIG. 11, according to various embodiments, the scissors assembly part C2 may include a first scissors slice D101, a second scissors slice D102, a spring plate bolt D103, a return spring D104, a reel D105, a bearing D106, a lock screw D107 (e.g. M1.2×5), a bracing wire D108, a lock nut D109 (e.g. M1.2), a fixed shell D110, a magnet D111 and a radio frequency identification (RFID) D112.

According to various embodiments, the motor assembly part C101 may be configured to produce a rotary motion by way of the motor D207 and to transmit the rotary motion of the motor D207 to the reel D105 of the scissors assembly part C2 by way of a motor output shaft D201. In other words, the motor D207 of the motor assembly part C101 may be configured to rotate the reel D105 of the scissors assembly part C2 via the motor output shaft D201. The reel D105 may be connected to the second scissors slice D102 via the bracing wire D108. According to various embodiments, one scissors slice (e.g. the first scissors slice D101) is immovable relative to the motorized needle holder. Accordingly, the other force slice (e.g. the second scissors slice D102) may be movable relative to the motorized needle holder. According to various embodiments, when the reel D105 is rotated in a first direction, the movable scissors slice (e.g. the second scissors slice D102) may be pulled towards the immovable scissors slice (e.g. the first scissors slice D101), and accordingly, a cutting function (or action) of the motorized microsurgery scissors is achieved.

Accordingly, according to various embodiments, a working principle of the motorized microsurgery scissors may be similar or identical to the working principle of the motorized needle holder in FIGS. 10A-10G.

FIG. 12A shows an exploded view of an injector (without a motor) according to various embodiments. FIG. 12B shows a side view of an injector (without a motor) according to various embodiments. FIG. 12C shows a cross-sectional side view of an injector (without a motor) according to various embodiments. As shown in FIGS. 12A-12C, the device is illustrated as an injector (without a motor).

As shown in FIG. 12A, the injector (without a motor) may include a glass syringe B101, a grip head B102, a first sealing washer B103, a second sealing washer B106, a first O ring B104, a second O ring B105, a second O ring B107, a fourth O ring B108, a catheter connector B109, a fixture B110 including a magnet, a XY assembly connector B111, a RFID B112 and a catheter B113.

According to various embodiments, working/technical principles of the disposable injector (without motor) are elaborated below.

Connection of the fixture B110 with XY assembly connector B111:

A terminal of the XY assembly connector B111 may include a ferromagnetic material. Accordingly, the fixture B110 and the XY deformation body may be magnetically coupled to each other.

Glass Syringe B101 Mounting:

As shown in FIG. 12A, the glass syringe B101 is inserted through the grip head B102 and through the first O ring B104, the second O ring B105, the first sealing washer B103 and through the second sealing washer B106. Further, an end of the glass syringe B101 is connected to the XY assembly connector B111. The first O ring B104 and the second O ring B105 are between the first sealing washer B103 and the second sealing washer B106 which are, in turn, between the grip head B102 and the XY assembly connected. Accordingly, when the glass syringe B101 is inserted through the grip head B102 and through the first O ring B104, the second O ring B105, the first sealing washer B103 and through the second sealing washer B106, and is further connected to the XY assembly connector B111, the first sealing washer B103 and the second sealing washer B106 may compress (e.g. squeeze) the first O ring B104 and the second O ring B105 therebetween, thereby creating a seal (e.g. fluid-tight) to seal the glass syringe B101 mounting.

Injection and Liquid Path:

One end of the catheter B113 may be connected to the XY assembly connector B111 through the catheter B113 connector B109, and another end of the catheter B113 may be mounted on an electric injector (another module). A path for fluid to flow between the electric injector and the glass syringe B101 may be provided (e.g. inside XY assembly connector), thereby enabling the electric injector and the glass syringe B101 to achieve fluid flow therebetween or fluid circulation.

FIG. 12D shows a perspective view of a motorized injector according to various embodiments. FIG. 12E shows a side view of a motorized injector according to various embodiments. FIG. 12F shows a cross-sectional side view of a motorized injector according to various embodiments. FIG. 12G shows a cross-sectional side view of a motorized injector according to various embodiments. FIG. 12H shows an exploded view of a motorized injector according to various embodiments. FIG. 12I shows a close-up view of a screw shaft of a motorized injector according to various embodiments. FIG. 12.1 shows a close-up view of a piston-nut of a motorized injector according to various embodiments.

As shown in FIGS. 12D-12H, the device is illustrated as a motorized injector.

The motorized injector may include a motor base B201, a motor B202, a bearing B203, a screw shaft B204, a jacket shell B205, a piston-nut B206, a glass tube B207, a plugging pipe B208, a first O seal ring B209, a second O seal ring B210, a fixed pin B211 and a glass syringe B212.

According to various embodiments, a transmission principle of the motorized injector may be based on a lead screw nut assembly which translates a rotary motion of the motor of a motor assembly part C101 into a rectilinear motion of the nut.

The lead screw nut assembly may include the screw shaft B204 and the piston-nut B206.

FIG. 12I shows the screw shaft B204. According to various embodiments, a first end 1 of the screw shaft B204 may be connected to the motor output shaft. For example, the first end 1 of the screw shaft B204 may include a recess 2 for receiving a portion (e.g. protrusion) of the motor output shaft. As shown, the recess 2 may comprise a tetrahedral shape (or other polyhedral shape) which may be configured to receive a portion of the motor output shaft a protrusion with a corresponding tetrahedral shape (or other polyhedral shape). A second end 3 of the screw shaft B204 may comprise a threaded exterior surface. The thread exterior surface of the second end 3 may be configured to receive and mate with a corresponding threaded interior surface of a recess 5 (in dotted lines) of the piston-nut B206, as shown in FIG. 12I, on a first portion 6 of the piston-nut B206.

Referring to FIGS. 12I and 12J, according to various embodiments, the first portion 6 of the piston-nut B206 may have a predetermined external non-circular shape. Further, the first portion 6 of the piston-nut B206 may be configured to fit within a through-hole (having a corresponding shape to receive the first portion 6) of the jacket shell B205, such that the piston-nut B206 is not rotatable relative to the jacket shell B205 but may slide within the jacket shell B205 along a longitudinal axis of the jacket shell B205. Accordingly, when the motor output shaft is connected to the first end 1 of the screw shaft B204, and when the second end 3 of the screw shaft B204 is inserted into (and mates with) the recess 5 of the piston-nut B206, and when the piston-nut B206 is within the jacket shell B205, a motor connected to the motor output shaft may rotate the second end 3 of screw shaft B204 within the recess 5 of the piston-nut B206 which causes the piston-nut B206 to move linearly (e.g. forward or backward) relative to the jacket shell B205 along the longitudinal axis of the jack shell. For example, a first rotation of the screw shaft B204 may cause the piston-nut B206 to move away from the screw shaft B204, and a second rotation of the screw shaft B204 (in an opposite direction from the first rotation) may cause the piston-nut B206 to move toward the screw shaft B204.

According to various embodiments, while the motorized injector in FIGS. 12D-12H is shown as an integral motorized injector (or non-detachable integral device), it may be envisioned that in various embodiments, the motorized injector may comprise a plurality of parts which may be detachably coupled to one another such that assembly and disassembly of a motorized injector may be performed easily and quickly by a user.

According to various embodiments, a method of forming a monitoring system to monitor a device may be provided. FIG. 13 is a schematic showing a method of forming a monitoring system according to various embodiments. The method of forming the monitoring system may include, in 1302, providing a magnification lens system to generate a magnified image of the device (e.g. magnified image of a tooltip of the device). The method of forming the monitoring system may further include, in 1304, providing at least three positional sensors in an arrangement around the magnification lens system to determine a position of the device.

According to various embodiments of the method of forming the monitoring system, the magnification lens system and the at least three positional sensors are attached to an overhanging arm extending from a first end portion of a stand. According to various embodiments, a base is attached to a second end portion of the stand.

According to various embodiments of the method of forming the monitoring system, a circular support is connected to the overhanging arm.

According to various embodiments of the method of forming the monitoring system, the circular support is configured to hold the at least three positional sensors.

According to various embodiments of the method of forming the monitoring system, the arrangement of the positional sensors is a circular arrangement.

According to various embodiments of the method of forming the monitoring system, the circular support is a rail.

According to various embodiments of the method of forming the monitoring system, the circular support includes a plurality of components which form the circular support.

According to various embodiments of the method of forming the monitoring system, the circular support includes a plurality of sensor supports attached to the circular support to hold the at least three positional sensors.

According to various embodiments of the method of forming the monitoring system, each sensor support of the plurality of sensor supports is attached to one positional sensor of the at least three positional sensors.

According to various embodiments of the method of forming the monitoring system, the positional sensors are arranged such that a first set of the at least three positional sensors, the first set including at least one positional sensor, lies in a first plane; and a second set of the at least three positional sensors, the second set including at least another positional sensor, lies in a second plane parallel to the first plane.

According to various embodiments of the method of forming the monitoring system, determining the position of the device is based on detection, by at least two of the at least three positional sensors, of light emitted from a plurality of light sources of the device.

According to various embodiments of the method of forming the monitoring system, the light is infrared light.

According to various embodiments of the method of forming the monitoring system, each positional sensor of the at least three positional sensors is configured to filter out a predetermined wavelength signal or a predetermined range of wavelength signals.

According to various embodiments of the method of forming the monitoring system, a visual indication of a working space of each positional sensor of the at least three positional sensors is provided by at least one visual indicator.

According to various embodiments of the method of forming the monitoring system, the device is a surgical tool, for example, a holder, single-blade cutter, dual-blade cutter or fluid injector.

According to various embodiments of the method of forming the monitoring system, the magnification lens system is a lens (e.g. magnifying lens), an optical microscope or a digital microscope.

A method of forming a surgical system may be provided. FIG. 14 is a schematic showing a method of forming a surgical system according to various embodiments. The method of forming the surgical system may include, in 1402, providing a monitoring system. The method of forming the surgical system may further include, in 1404, providing a device.

According to various embodiments of the method of forming the surgical system, the device may include a plurality of light sources.

According to various embodiments of the method of forming the surgical device, the device may be a surgical tool, for example, a holder, single-blade cutter, dual-blade cutter or fluid injector.

According to various embodiments, the method of forming the surgical system further includes providing a further device comprising a plurality of light sources.

According to various embodiments, the method of forming the surgical system further includes providing an image detector configured to detect the magnified image generated by the magnification lens system.

According to various embodiments, the method of forming the surgical system further includes providing a monitor coupled to the image detector.

According to various embodiments, the method of forming the surgical system further includes providing a computer configured to receive a first data on the position of the device and a second data on the position of the further device, from at least two of the at least three positional sensors of the monitoring system.

According to various embodiments, the method of forming the surgical system further includes providing a controller configured to control the device and the further device based on the first data and the second data.

While the invention has been particularly shown and described with reference to specific embodiments, it should be understood by those skilled in the art that various changes in form and detail may be made therein without departing from the spirit and scope of the invention as defined by the appended claims. The scope of the invention is thus indicated by the appended claims and all changes which come within the meaning and range of equivalency of the claims are therefore intended to be embraced.

1. A monitoring system for monitoring a device, the system comprising:
a magnification lens system configured to generate a magnified image of the device; and
at least three positional sensors in an arrangement around the magnification lens system;
wherein the at least three positional sensors are configured to determine a position of the device.

2. The monitoring system of claim 1, further comprising:
a stand having a first end portion and a second end portion;
an overhanging arm extending from the first end portion of the stand; and
a base attached to the second end portion of the stand;
wherein the magnification lens system and the at least three positional sensors are attached to the overhanging arm.

3. The monitoring system of claim 2, further comprising:
a circular support connected to the overhanging arm;
wherein the circular support is configured to hold the at least three positional sensors; and
wherein the arrangement of the at least three positional sensors is a circular arrangement.

4. The monitoring system of claim 3, wherein the circular support is a rail.

5. The monitoring system of claim 3, wherein the circular support comprises a plurality of components which form the circular support.

6. The monitoring system of claim 3, further comprising:
a plurality of sensor supports attached to the circular support to hold the at least three positional sensor;
wherein each sensor support of the plurality of sensor supports is attached to one positional sensor of the at least three positional sensor.

7. The monitoring system of claim 1, wherein the at least three positional sensors are arranged such that a first set of the at least three positional sensors, the first set comprising at least one positional sensor, lies in a first plane; and a second set of the at least three positional sensors, the second set comprising at least another positional sensor, lies in a second plane parallel to the first plane.

8. The monitoring system of claim 1, wherein determining the position of the device is based on detection, by at least two of the at least three positional sensors, of light emitted from a plurality of light sources of the device.

9. The monitoring system of claim 8, wherein the light is infrared light.

10. The monitoring system of claim 8, wherein each positional sensor of the at least three positional sensors is configured to filter out a predetermined wavelength signal or a predetermined range of wavelength signals.

11. The monitoring system of claim 1, further comprising:
a plurality of visual indicators;
wherein at least one visual indicator of the plurality of visual indicators is configured to provide a visual indication of a working space of a positional sensor of the at least three positional sensors.

12. The monitoring system of claim 1, wherein the magnification lens system is a lens, an optical microscope or a digital microscope.

13. A surgical system comprising:
the monitoring system of claim 1; and
the device.

14. The surgical system of claim 13, wherein the device comprises a plurality of light sources; and
wherein a position of the device is determined, by at least two of the at least three positional sensors of the monitoring system, based on light emitted from the plurality of light sources of the device.

15. The surgical system of claim 14, wherein the device is a surgical tool.

16. The surgical system of claim 15, wherein the surgical tool is a holder, single-blade cutter, dual-blade cutter or fluid injector.

17. The surgical system of claim 14, further comprising:
a further device comprising a plurality of light sources; and
wherein a position of the further device is determined, by at least two of the at least three positional sensors of the monitoring system, based on light emitted from the plurality of light sources of the further device.

18. The surgical system of claim 13, further comprising:
an image detector configured to detect the magnified image generated by the magnification lens system; and
a monitor coupled to the image detector.

19. The surgical system of claim 17, further comprising:
a computer configured to receive a first data on the position of the device and a second data on the position of the further device; and
a controller configured to control the device and the further device based on the first data and the second data.

20. A method of forming a monitoring system to monitor a device, the method comprising:
providing a magnification lens system to generate a magnified image of the device; and
providing at least three positional sensors in an arrangement around the magnification lens system to determine a position of the device.

21. The method of claim 20,
wherein the magnification lens system and the at least three positional sensors are attached to an overhanging arm extending from a first end portion of a stand; and
wherein a base is attached to a second end portion of the stand.

22. The method of claim 21,
wherein a circular support is connected to the overhanging arm;
wherein the circular support is configured to hold the at least three positional sensors; and
wherein the arrangement of the positional sensors is a circular arrangement.

23. The method of claim 22,
wherein the circular support is a rail.

24. The method of claim 22,
wherein the circular support comprises a plurality of components which form the circular support.

25. The method of claim 22,
wherein a plurality of sensor supports are attached to the circular support to hold the at least three positional sensors; and
wherein each sensor support of the plurality of sensor supports is attached to one positional sensor of the at least three positional sensors.

26. The method of claim 20,
wherein the positional sensors are arranged such that a first set of the at least three positional sensors, the first set comprising at least one positional sensor, lies in a first plane; and a second set of the at least three positional sensors, the second set comprising at least another positional sensor, lies in a second plane parallel to the first plane.

27. The method of claim 20,
wherein determining the position of the device is based on detection, by at least two of the at least three positional sensors, of light emitted from a plurality of light sources of the device.

28. The method of claim 27,
wherein the light is infrared light.

29. The method of claim 27,
wherein each positional sensor of the at least three positional sensors is configured to filter out a predetermined wavelength signal or a predetermined range of wavelength signals.

30. The method of claim 20,
wherein a visual indication of a working space of each positional sensor of the at least three positional sensors is provided by at least one visual indicator.

31. The method of claim 20,
wherein the device is a surgical tool.

32. The method of claim 31,
wherein the surgical tool is a holder, single-blade cutter, dual-blade cutter or fluid injector.

33. The method of claim 20,
wherein the magnification lens system is a lens, an optical microscope or a digital microscope.

34. A method of forming a surgical system, the method comprising:
providing the monitoring system of claim 1; and
providing the device.

35. The method of claim 34,
wherein the device comprises a plurality of light sources.

36. The method of claim 35,
wherein the device is a surgical tool.

37. The surgical system of claim 36,
wherein the surgical tool is a holder, single-blade cutter, dual-blade cutter or fluid injector.

38. The method of claim 34, further comprising:
providing a further device comprising a plurality of light sources.

39. The method of claim 34, further comprising:
providing an image detector configured to detect the magnified image generated by the magnification lens system; and
providing a monitor coupled to the image detector.

40. The method of claim 38, further comprising:
providing a computer configured to receive a first data on the position of the device and a second data on the position of the further device, from at least two of the at least three positional sensors of the monitoring system; and
providing a controller configured to control the device and the further device based on the first data and the second data.
